(19) United States
(12) Patent Application Publication
(10) Pub. No.: US 2019/0302069 A1
(43) Pub. Date: Oct. 3, 2019

(54) APPARATUS AND METHOD FOR CLASSIFYING A TOBACCO SAMPLE INTO ONE OF A PREDEFINED SET OF TASTE CATEGORIES

(71) Applicant: BRITISH AMERICAN TOBACCO (INVESTMENTS) LIMITED, London (GB)

(72) Inventors: Oscar Francisco Swenson Pontes, Centro Rio de Janeiro (BR); Guilherme Post Sabin, Centro Rio de Janeiro (BR); Jose Roberto Pereira da Silva, Porto Alegre (BR); Jailson Cardoso Dias, Centro Rio de Janeiro (BR); Samuel Kaiser, Centro Rio de Janeiro (BR)

(21) Appl. No.: 16/315,436

(22) PCT Filed: Jun. 27, 2017
(86) PCT No.: PCT/GB2017/051870

§ 371 Date: Jan. 4, 2019

(30) Foreign Application Priority Data
Jul. 4, 2016 (GB) 1611596.6

(51) Int. Cl. G01N 30/86; G01N 30/72; G01N 33/00

(52) U.S. Cl. CPC G01N 30/8675; A24B 15/10; G01N 33/0098; G01N 30/7233

(57) ABSTRACT

A method and apparatus are provided for classifying a tobacco sample of a particular tobacco type into one of a predefined set of taste categories for that tobacco type. The method comprises acquiring mass spectrometry data from the tobacco sample; identifying from the acquired mass spectrometry data a plurality of chemical components and their respective content levels within the tobacco sample; and assigning the tobacco sample to one of the predefined set of taste categories for that tobacco type based on the plurality of chemical components and their respective content levels identified within the tobacco sample, using a statistical multivariate regression model that represents a relationship between the chemical components and the taste categories.

### FIELD

The present disclosure relates to a method and apparatus for classifying a tobacco sample into one of a predefined set of taste categories based on the chemical components and their respective content level within the tobacco sample.

### BACKGROUND

Tobacco is an agricultural crop of considerable economic importance, used primarily in the manufacture of cigarettes, cigars, and other such products. Tobacco is grown in more than one hundred (mostly tropical) countries, spread across North and South America, Europe, Africa and Asia, including, for example, Brazil, Italy, Turkey, Pakistan, USA and Tanzania. There are various types (varieties) of tobacco, the three most common types being Virginia, grown frequently in countries like Brazil, China, India, Tanzania and the US; Burley, grown frequently in countries like Brazil, Italy and the US; and Oriental, grown frequently in countries like Greece and Turkey. Virginia tobacco is usually cured in heated barns (so is sometimes referred to as “flue-cured tobacco), Burley is usually air-cured in barns, while Oriental is usually sun-cured in the open air. Cigarettes may be produced containing just one variety of tobacco, e.g. Virginia, or blends of multiple varieties of tobacco.

The consumer experience of tobacco typically occurs through smoking a cigarette or cigar, and is characterised by various sensory inputs relating to flavour, taste, aroma, etc. Various attempts have been made break down a given flavour or taste into a number of factors or parameters, such as bitterness, dryness, etc. The factors then form a multi-dimensional measurement system for assessing tobacco taste from a consumer perspective. However, because such factors are semi-qualitative in nature, they are generally assessed by people smoking the tobacco, which makes reproducibility more difficult.

Cigarette manufacturers typically want to provide consumers with a consistent and reliable product, including in terms of the various sensory factors mentioned above. It can be challenging to achieve such consistency, given that tobacco is a natural product. Thus the tobacco is subject to intrinsic variation between individual plants, combined with additional variations caused by differences in growing location, soil, etc. Indeed, even tobacco grown at a single location may experience fluctuations in properties, for example, based on changes in climate (e.g. whether the growing period has been relatively hot and dry or cold and wet) and/or details of subsequent processing for the tobacco (such as curing). These difficulties may be compounded by having multiple different varieties in a tobacco blend, although on the other hand, a blend is often performed to try to compensate for such variation.

In practice, the manufacturers of tobacco products frequently rely on the human expertise and experience to acquire the tobacco leaf and form the blends that will produce the desired sensory characteristics for a given brand of cigarette (or other tobacco product). Usually, this procedure then involves confirmation by test smoking of the resulting cigarettes that the resulting product does indeed have the desired sensory characteristics, and if not, further refinement of the blend may have to be performed.

A further consideration is that tobacco material is now being used in a new generation of devices, in which the tobacco material (including derivatives thereof) is heated to create a vapour (as opposed to being burned to create smoke, as in conventional cigarettes). Such new generation devices, which are sometimes referred to as vaping devices, include various types of e-cigarettes that typically use battery power to heat the tobacco material. Compared with conventional cigarettes, such vaping devices are relatively new, have a wide range of designs, and may utilise the tobacco material in a number of different forms, including as a paste, a dried powder, a liquid extract, dried leaves, fresh leaves etc. Accordingly, it can be relatively difficult to predict the sensory outcome resulting from any given choice of tobacco material in such devices.

### SUMMARY

The invention is defined in the appended claims.

Various embodiments of the invention provide an apparatus and a corresponding method for classifying a tobacco sample of a particular tobacco type into one of a predefined set of taste categories for that tobacco type. The method comprises acquiring mass spectrometry data from the tobacco sample; identifying from the acquired mass spectrometry data a plurality of chemical components and their respective content levels within the tobacco sample; and assigning the tobacco sample to one of the predefined set of taste categories for that tobacco type based on the plurality of chemical components and their respective content levels identified within the tobacco sample, using a statistical multivariate regression model that represents a relationship between the chemical components and the taste categories. Various embodiments of the invention further provide a method for generating such a statistical multivariate regression model.

### BRIEF DESCRIPTION OF THE DRAWINGS

Various embodiments of the invention will now be described in detail by way of example only with reference to the following drawings:

FIG. 1 is a schematic diagram of a typical global workflow for a metabolomic analysis as described herein.

FIG. 2 is a schematic diagram showing a summary of the extraction and LC analysis procedures used for the untargeted analysis described herein.

FIG. 3 is a table showing the UPLC-HDMS^{E} parameters employed for the metabolomic analysis of the Virginia tobacco.

FIG. 4 provides representative ion maps obtained from the analysis of blend (A—left) and smoke (B—right) from use of the semi-polar UPLC-HDMS^{E} method described herein. (The lower plot for each case represents the full data set, the upper plot represents a zoom of a specific region of the ion map corresponding to a subset of the full data set).

FIG. 5A is a plot showing results from the OPLS-DA model for the various taste blends having certain standard tastes, while FIG. 5B is a plot showing results from the OPLS-DA model for the various internal grades of leaf.

FIG. 6 is an analogous plot to FIG. 5B, but the different internal grades are coloured in the plot based on leaf colour (rather than on taste as in FIG. 5B).

FIG. 7 is a table that provides an overview of the main results estimated by the OPLS-DA models for the blend results from analysis of the three taste blends as described herein.

FIG. 8 shows the content level of various chemical families for each of the three tastes for the blend results, with plot A (left) showing the major chemical components, and plot (B) showing the more minor chemical components.

FIG. 9 is a plot which shows the content level of Maillard reaction products and free carbohydrates for each of the 3 tastes T1, T2 and T3.

FIG. 10A is a plot showing results from the OPLS-DA model for the various taste blends from the standard smoke analysis, while FIG. 10B is a plot showing results from the OPLS-DA model for the various internal grades.

FIG. 11 is an analogous plot to FIG. 10B, but the different internal grades are coloured in the plot based on leaf colour (rather than on taste as in FIG. 10B).

FIG. 12 is a table that provides an overview of the main results estimated by the OPLS-DA models for the smoke results from analysis of the three taste blends as described herein.

FIG. 13 shows the content level of various chemical families for each of the three tastes for the smoke results, with plot A (left) showing the major chemical components, and plot (B) showing the more minor chemical components.

FIG. 14 shows the result of applying an O2PLS model to generate separate representations of the blend samples (A—left) and smoke samples (B—right) for the internal grades of Virginia tobacco.

FIG. 15 shows a correlation performed using the O2PLS model between the blend results from FIG. 14A and the smoke results from FIG. 14B.

FIG. 16 shows the result of applying an O2PLS model to generate separate representations from the blend analysis by UPLC-HDMS^{E} methodology (A—left) and HTS-FIA-HRMS methodology (B—right) for the internal grades of Virginia tobacco.

FIG. 17 shows a correlation performed using the O2PLS model between UPLC-HDMS^{E} results from FIG. 16A and the HTS-FIA-HRMS results from FIG. 16B.

FIG. 18 is a detailed flow-chart showing one approach for developing the multivariate models described herein.

FIG. 19 illustrates a hierarchical decision tree for use in grading tobacco as described herein.

FIG. 20 is a table of results from a blind validation of a tobacco grading tool developed from HTS-FIA-HRMS analysis.

FIG. 21 is a plot illustrating a comparison between theoretical sensorial attributes of smoke from pure tobacco (blue) and the predicted sensorial attributes (red), the latter being obtained from a tool developed from HTS-FIA-HRMS analysis.

FIG. 21A is a plot illustrating a comparison between the theoretical sensorial attributes of smoke from a particular cigarette brand (blue) and the predicted sensorial attributes (red), the latter being obtained from a tool developed from HTS-FIA-HRMS analysis.

FIG. 21B is a plot illustrating a comparison between the theoretical sensorial attributes of vapour from a heat-not-burn device (blue) and the predicted sensorial attributes (red), the latter being obtained from a tool developed from HTS-FIA-HRMS analysis.

FIG. 22 illustrates a plot used for recognising samples with innovative and/or enhanced taste from the HTS-FIA-HRMS methodology described herein, where the Y axis (vertical) represents the innovative taste score and the X axis (horizontal) represents the enhancement taste score.

FIG. 23 shows a dendrogram showing samples clustered in accordance with their global chemical composition determined by HTS-FIA-HRMS analysis.

FIG. 24 illustrates the fitting of a multivariate model for estimating the quality crop index (QCI) from HTS-FIA-HRMS analysis.

FIG. 25 shows the fitting of a multivariate model for estimating the nicotine content (A—upper) and total sugar content (B—lower) from HTS-FIA-HRMS analysis.

### DETAILED DESCRIPTION

### 1) Overview

Although advanced analytical techniques are improving year after year, the characterization of compounds in highly complex natural products remains a challenge. In this context, the complexity of tobacco and its physical and chemical properties derives from the large number of chemical classes present in smoke (or vapour), the formation of blends, and the relationship between the compounds present in blend compared with the sensorial properties of the resulting smoke (or vapour). Tobacco chemical variability is influenced by factors including polarity, solubility, volatility, and thermal stability, among others.

Strategies for metabolomic analysis in general, according to reports published in Nature Protocols (De Vos et al. 2007), often comprise four steps:

Step 1—Extraction: an untargeted approach is carried out using a few procedures—typically three, considering the chemical polarity of compounds, e.g. an extraction procedure for polar, another extraction procedure for semi-polar, and another one for nonpolar.
Step 2—Instrumental analysis: there is no single separation technology available at present which is capable of covering all types of categories of compounds. Accordingly, as for step 1, multiple different separation procedures may be utilised.
Step 3—Data analysis: when analytical information is acquired from an untargeted analysis, a very large volume of data may be generated, which can then require a correspondingly large time for processing.
Step 4—Modeling: for untargeted analysis, this is possibly the most significant step, because the content of information may be highly complex, as well as often being partially or fully unknown in terms of structure. Accordingly, it may require a long period of time for building, optimizing and performing iterations on the original data in order to derive a suitable model.

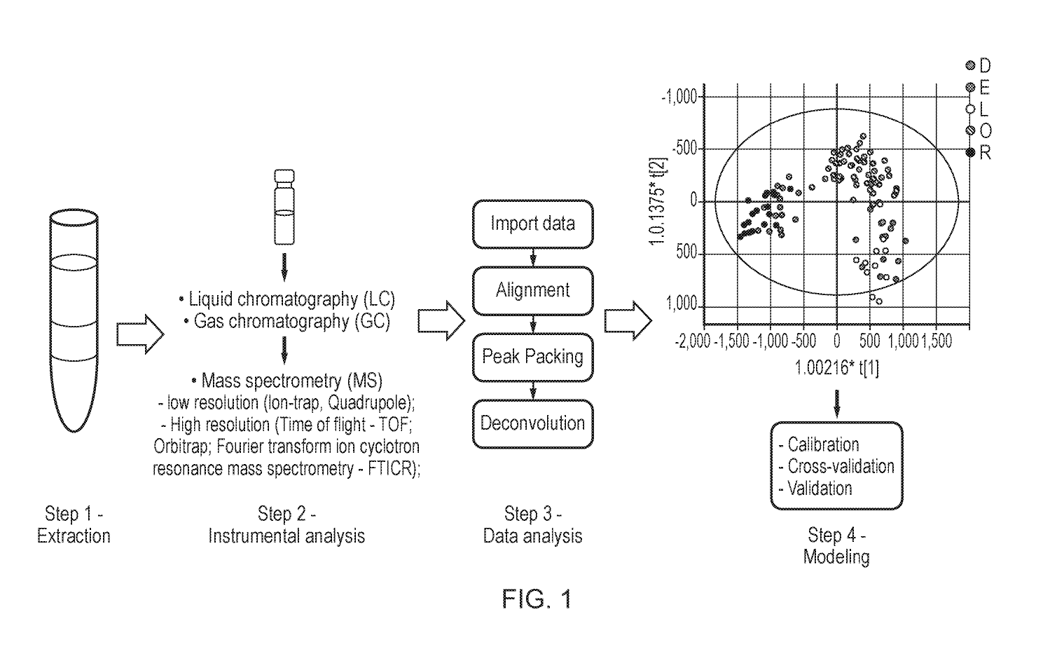

The above approach is illustrated in FIG. 1, which shows a typical global workflow for metabolomic analysis starting with an extraction procedure (Step 1), definition of instrumental approach for chemical analysis (Step 2), data processing of the instrumental results (Step 3) and generation/assessment of model(s) (Step 4). Such a workflow is indicated in various reference procedures and international protocols of metabolomic analysis (Fiehn et al. 2000; Kim & Verpoorte 2010; Villas-Boas et al. 2005; De Vos et al. 2007).

Liquid-solid extraction (LSE) is the technique most widely used to transfer compounds from a matrix (such as tobacco leaf) to solvent (step 1 of FIG. 1). The extract is obtained by mixing and shaking the solid phase with one or more solvents such that physical interaction takes place and mass is transferred to liquid phase (by different mechanisms such as diffusion, dissolution, desorption). The choice of solvent system (binary, tertiary, and pH) is important so that it is capable of extracting a large number of compounds with high reproducibility despite matrix variations (moisture, average particle size, etc). Often, to achieve the most complete profile of extraction, segmentation into different solvent systems is utilised.

Compounds found in tobacco blend and smoke may have high molecular weight, for example, fatty acids, triacylglycerols, esters, phospholipids, carbohydrates, or lower molecular weight, such as amino acids, organic acids, and pyrazines. Liquid chromatography (LC) combined with mass spectrometry (MS) is an instrumental approach (step 2 of FIG. 1) which is suited for the analysis of compounds with a large range of molecular weight and polarity in a single matrix (Villas-Boas et al. 2005; De Vos et al. 2007; Theodoridis et al. 2011).

In chromatography, variation in the stationary phase is usually performed using a range from hydrophilic phases to reverse phase, allowing for each extraction protocol scope (Gama et al. 2012; McCalley 2010; De Vos et al. 2007). Besides chromatography, one new technology for mass spectrometry which has shown significant advantages is ion mobility spectrometry (IMS). This technology has the capability of separating ions of the same m/z ratio (the conventional measured parameter detected in mass spectrometry), but with different collision cross-sections (CCS) and/or charge states by monitoring the mobility of an ion in a gaseous chamber under the influence of an electric field. Thus, the IMS provides an extra degree of analytical opportunity for conformational ensembles of compounds with equivalent m/z. The advantages of IMS include separation of isomers, isobars, and conformers; reduction of chemical noise; and also measurement of ion size. Applications of IMS range from investigations in various “omics” fields (e.g. genomics, proteomics or metabolomics) to quantitative analysis, including for inorganic, organometallic, and even intact proteins (Shvartsburg et al. 2004; Viehland et al. 2000).

The application of the approach of FIG. 1 for tobacco analysis expands previous work in this area, including by performing an untargeted analysis of tobacco metabolomics. In other words, rather than starting with a set of pre-identified compounds, and then using techniques or measurements strategies especially targeted at such pre-identified compounds, the approach described herein seeks to investigate, measure and analyse compounds that are present in a given tobacco sample and which form a representative range of the tobacco metabolome. This more extensive scope of investigate has been found to provide more powerful models (as per step 4 of FIG. 1) for use in predicting the sensory properties of tobacco (and then to support the utilization of such models).

As part of the work described herein, blend and smoke analyses of Virginia tobacco have been performed using UPLC-HDMS^{E} (ultra performance liquid chromatography, high definition mass spectrometry). (The “^{E}” following the HDMS is used to indicate a form of tandem mass spectrometry data acquisition using both low and high energy collision-induced dissociation, which are used to obtain accurate masses for the precursor and product ions respectively). Analytical data were processed (step 3 of FIG. 1) by using the Progenesis QI software product from Nonlinear Dynamics (see http://www.nonlinear.com/progenesis/qi/), which provides molecule discovery analysis software for LC-MS data, and also the SIMCA software from MKS Data Analytics Solutions (see http://umetrics.com/products/simca), which is a multivariate data analysis tool, including for use with spectroscopic data. The chemometrics modeling (step 4 of FIG. 1) was performed using software created with the PLS toolbox (PLS=partial least squares) from Eigenvector Research Incorporated (see http://www.eigenvector.com/software/pls_toolbox.htm), which provides various chemometric multivariate analysis tools, and which is used in conjunction with the MATLAB computational environment from MathWorks (see http://uk.mathworks.com/products/matlab/). (It will be appreciated that these various analysis and computational techniques and products are provided by way of example only, and other corresponding facilities may be used as appropriate).

The approach described herein can be integrated into a High Throughput Screening (HTS) analysis. Such integration helps to increase the analytical capability described herein, and supports the use of this technology across a wider range of applications (as described in more detail below).

### 2) Experimental Procedure

LC-MS grade methanol (MeOH), acetonitrile (ACN), chloroform, and formic acid (FA) were obtained from Merck (Darmstadt, Germany), and ultra-pure water was produced by a Milli-Q apparatus (Millipore®, Billerica, Mass., USA). All materials used were carefully washed using LC grade solvents and/or ultra-pure water produced by the Milli-Q apparatus. In view of the high sensitivity of the mass analyzers, surfactants and similar products were not used in the washing procedures in order to avoid damage to the instruments, and also (in particular) to avoid cross-contamination between the instruments. For similar reasons, the reagents and samples were handled using chemical-resistant, powder-free gloves. Polyalanine (1 μg/mL), sodium formate (0.5 mmol/L), and leucine encephalin (1 μg/mL), obtained from Waters Reference Solutions (USA) were used for collision cross-section calibration, mass calibration, and lock mass (i.e. known m/z) correction, respectively. All solutions were prepared on the day of procedure.

The main parameters influencing the quality of an extract are the plant parts used as starting material, the physical properties of the bulk material (e.g. particle size, moisture), the solvent system used for extraction, and the extraction technology (operations and equipment). The procedure adopted for this experiment was based on international reference methodologies for metabolomics untargeted analysis (De Vos et al. 2007; Theodoridis et al. 2011), having regard also to various other factors, including particular features related with tobacco matrix, tobacco sample type (smoke and blend), cost-effectiveness. A further objective was to maximize the number of compounds that can be determined from a single portion of extraction, thereby allowing the resulting chemometric models to be as specific and representative as possible. The experimental protocol utilized impartial selection, whereby the choice of the order of the experimental units for extraction procedures and UPLC-HDMS^{E} run was randomised. In order to control extraction procedure variability, three samples were extracted from a given material and named as extract controls (EC1, EC2, and EC3). In addition, the system performance throughout the sample set was monitored by reanalysis of the same sample after twenty analyses for both smoke and blend.

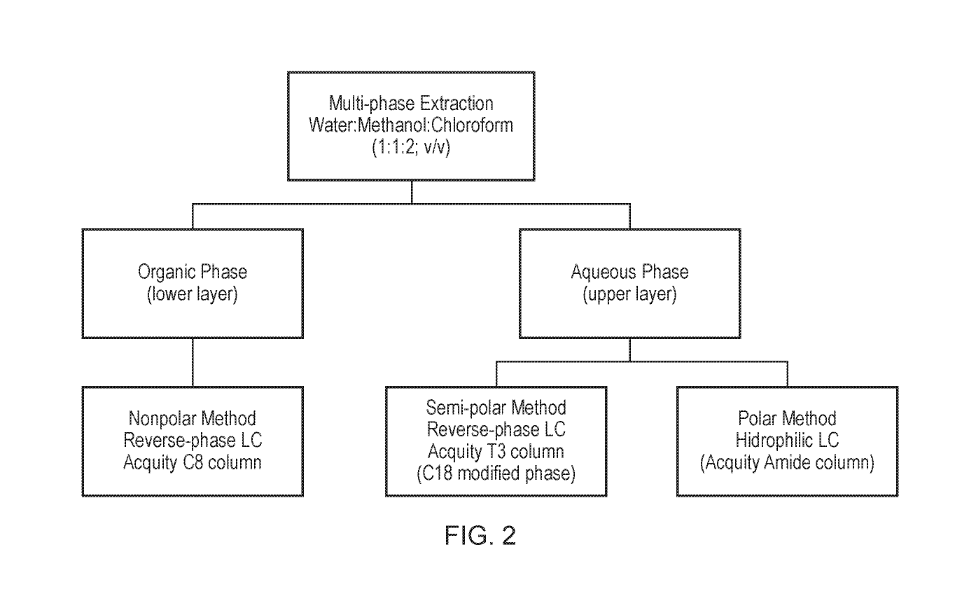

FIG. 2 presents a summary of the extraction and LC analysis procedures used for untargeted analysis described herein for both (tobacco leaf) blend and smoke. In essence, there is a multi-phase extraction performed using a combination of three solvents (water, methanol and chloroform). This then produces an organic phase (lower layer), which is then subjected to a nonpolar method for LC, and an aqueous phase (upper layer), which is then subject to each of a polar method and a semi-polar method for LC.

For the blend extraction procedure, aliquots of 200 mg of various powdered samples of Virginia tobacco (crop 2013) that had been sensorially characterized were used. A total of 142 samples were used, each sample having been classified into one of 3 sets of taste characteristics (denoted for convenience herein as T1, T2 and T3). 110 of the samples had been subject to detailed internal grading, including allocation to the taste sets: 27 samples of T1, 52 samples of T2, and 33 samples of T3. The remaining 30 samples had not been subject to such internal grading, but nevertheless had been blended to one of the same three taste sets: 10 samples each of T1, T2 and T3.

The samples were transferred to centrifuge tubes of 20 mL and extracted with 5 mL of methanol:water solution (1:1,v/v; aqueous phase) plus 5 mL of chloroform (organic phase), placed in a sonicator for 15 min, followed by shaking at 250 rpm for 15 min. Then, centrifugation was performed at 2500 rpm for 5 min. Aliquots of 2 mL of aqueous phase (upper layer) and organic phase (lower layer) were filtered through a 0.22 μm filter (Millipore, USA), diluted (20 times) and transferred to respective vials for UPLC-HDMS^{E} analysis.

Cigarettes were manufactured using Virginia tobacco (crop 2013) based on the same sample sets as described above for the powdered samples—112 cigarette samples categorized by the internal grading (27 of T1, 52 of T2, and 33 of T3), and 30 samples which had not been graded, but formed from the taste blends categorized as T1, T2 or T3 (10 of each). The cigarettes were conditioned at 22±1° C. and 60±3% relative humidity for 48 hours prior to smoking so as to maintain their physical equilibrium. Each set of 5 cigarettes was smoked using a Cerulean SM 450 smoking machine (see http://www.cerulean.com/product-services/tobacco/smoking-machines) under the standard smoking regime, one puff per min, 2 s puff duration, 35 mL puff volume (ISO 3308, 2012). The particulate phase smoke of the set of 5 cigarettes was collected on a 44 mm Cambridge filter pad (see http://www.cambridgefilterusa.com/) and transferred to a 50 mL erlenmeyer flask and extracted with 10 mL of methanol:water solution (1:1,v/v; aqueous phase) plus 10 mL of chloroform (organic phase) by shaking at 250 rpm for 30 min. Aliquots of 2 mL of aqueous phase (upper layer) and organic phase (lower layer) were filtered through a 0.22 μm filter (Millipore, USA), diluted (2 times for aqueous phase and 20 times for organic phase) and transferred to respective vials for UPLC-HDMS^{E} analysis.

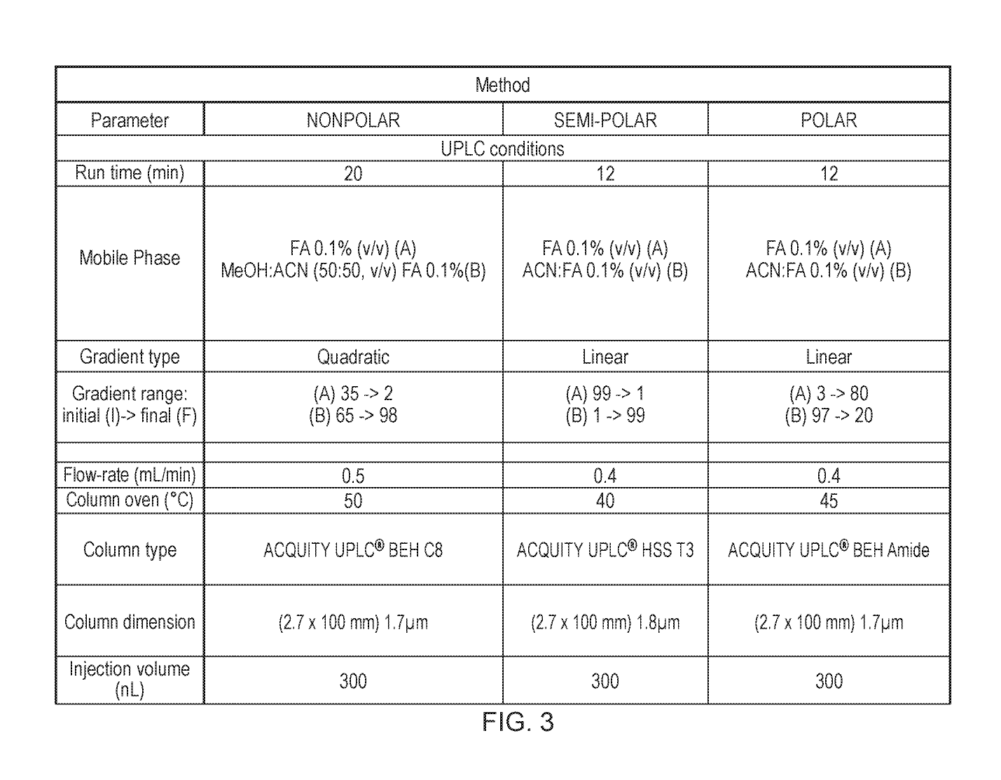

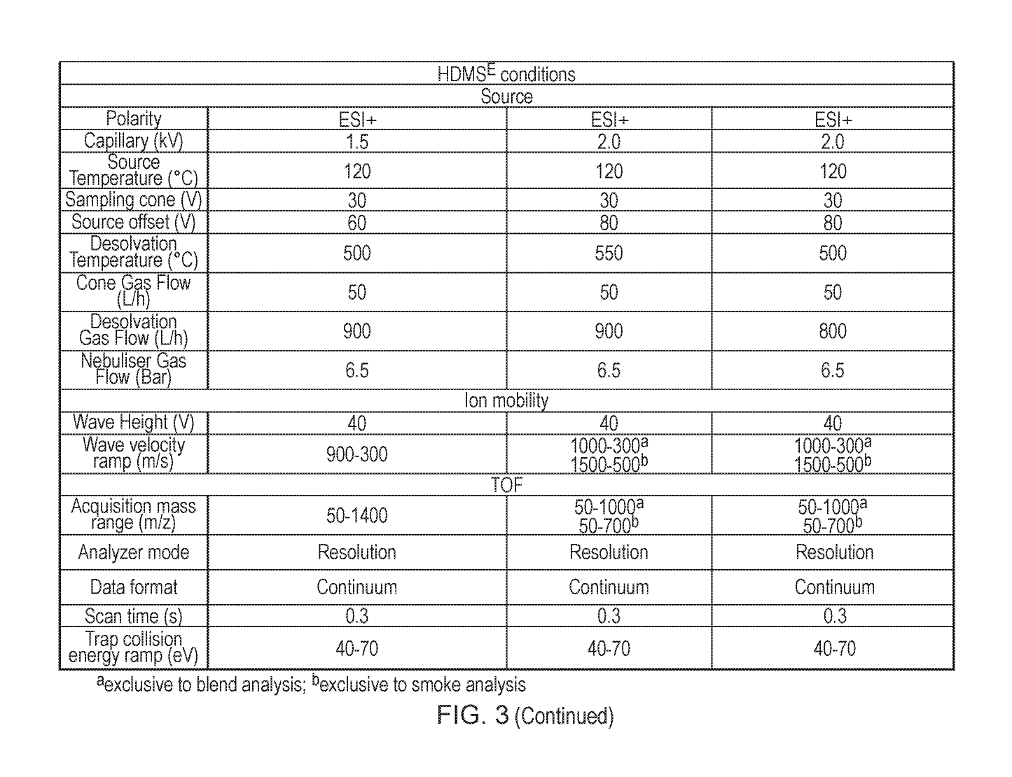

As shown in FIG. 2, three independent UPLC methods were employed for the tobacco metabolomic analysis, namely, nonpolar, semi-polar and polar methods (see also FIG. 1). All analyses were performed using an ACQUITY I-CLASS UPLC module coupled with an SYNAPT G2-Si HDMS (both Waters, USA—see http://www.waters.com/). An appropriate system check (detector setup, mass calibration, and collision cross-section calibration) was performed before each analysis batch. The data acquisition was performed at positive MS^{E} resolution mode employing ion mobility (HDMS^{E}). The MS^{E} mode allows one to obtain both a low energy spectrum and a high energy spectrum from the same run without discrimination or ion pre-selection. Nitrogen was used as nebulizer, cone, desolvation, and drift gas for ion mobility. Argon was used as the collision gas. The UPLC-HDMS^{E} parameters employed for the metabolomic analysis of the Virginia tobacco are set out in the table of FIG. 3.

### 3) Data Processing and Chemometric Strategy

In order to investigate the potential chemical markers responsible for the differentiation the T1, T2 and T3 tastes (for both blend and smoke) a new chemometric strategy was developed and applied. All the raw data from UPLC-HDMS^{E} analyses were processed using the Progenesis QI software (as mentioned above) according to the following workflow: importing the raw data; m/z and time alignment; choice of experimental design (from among objects in this case); peak-picking and normalization; and deconvolution (for more details of these operations, see the Progensis QI User Guide 1.0, available at http://storage.nonlinear.com/webfiles/progenesis/qi/v1.0/user-guide/Progenesis_QI_User_Guide_1.0.pdt). The resulting X and Y matrices (for blend and smoke respectively) were exported as CSV file (comma separated variable) and processed in a high-level technical computer language (MATLAB, as mentioned above) by using high specification computers (192 GB of RAM). An advanced automated chemometric system (ACS) was established and applied to the high-resolution MS datasets.

The data calibration and prediction steps were performed using a multivariate regression model based on Orthogonal Partial Least Squares Discriminant Analysis (OPLS-DA) using Pareto scaling (scaling by the square root of the standard deviation) and mean center preprocessing methods. For cross-validation, the Venetian blind method was employed for a calibration set of 20 samples having ten data splits and one sample per blind. This approach reassigns randomly selected blocks of data in order to determine the Root Mean Square Error of Cross-Validation (RMSECV) for the model. In order to estimate the Root Mean Square Error of Prediction (RMSEP), 21 samples were used for the calibration set and 9 samples (both randomly selected) were used for the prediction set.

In order to determine the total correlation between blend and smoke, a matrix X (blend) and a matrix Y (smoke) obtained from analyses of the internally graded samples were exported to SIMCA software (Umetrics, Sweden) after the processing in the Progenesis QI as described above. The correlation was derived based on an O2PLS model (two-way orthogonal PLS), again using Pareto scaling and mean center preprocessing. These results were the verified based on internal validation (cross-validation), external validation, and a response permutation test was performed. Observations with a distance to model (DModX) higher than 2 were defined as outliers.

In order to identify the chemical nature of the compounds responsible for the differentiation in the three taste groups (T1, T2 and T3), the exact mass and isotopic patterns of chemical markers obtained from OPLS-DA models were compared with high resolution mass libraries. This comparison was performed using the MetaScope plug-in for the Progenesis QI software mentioned above (again from Nonlinear Dynamics). In addition, the theoretical fragmentation pattern obtained from the hits was compared to experimental data (high energy spectrum). Thresholds of 10 ppm error in relation to exact mass and 80% for isotopic pattern similarity were set for search in the libraries. Standard compounds, when available, were analyzed, for structure confirmation by comparison between retention and mass spectrum (high energy spectrum) of standard compounds with unknown compounds.

### 4) Results

### a) UPLC-HDMS^{E} Analysis

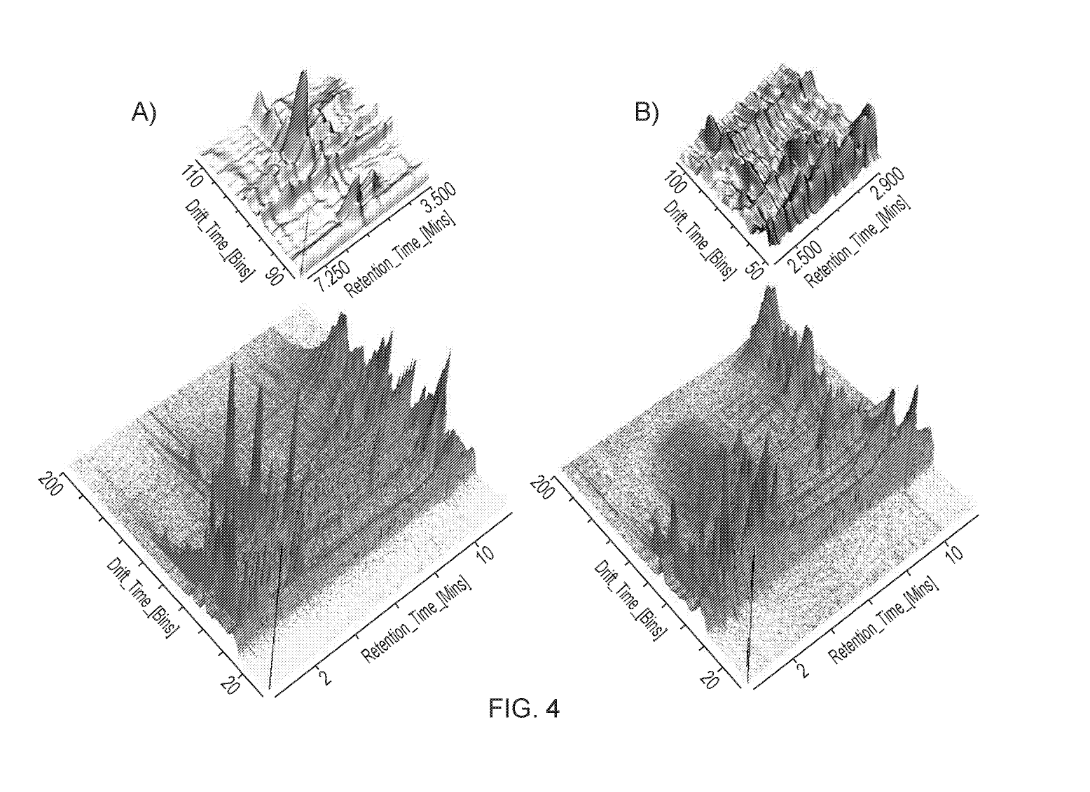

Representative ion maps obtained from blend and smoke analysis are shown in FIG. 4.

The maps to the left relate to the blend (A), while the maps to the right relate to the smoke (B), in both cases obtained from the semi-polar UPLC-HDMS^{E} method (see FIG. 2). For both (A) and (B), the lower plot shows the complete data set of drift time (bins) against retention time (minutes), while the upper plot is a higher resolution diagram of a portion of the same data set (i.e. for a subset of the range of drift time and retention time). More than twenty thousand ions were detected for each data matrix (blend and smoke) after data processing in Progenesis QI software, which shows that the UPLC-HDMS^{E} methods (nonpolar, semi-polar and polar) are able to detect an extensive range of total metabolites present in tobacco. It was confirmed by various control procedures that the results obtained were not significantly affected by extraction procedure variability or instability in the system performance throughout the analysis of the sample set.

### b) Blend Analysis

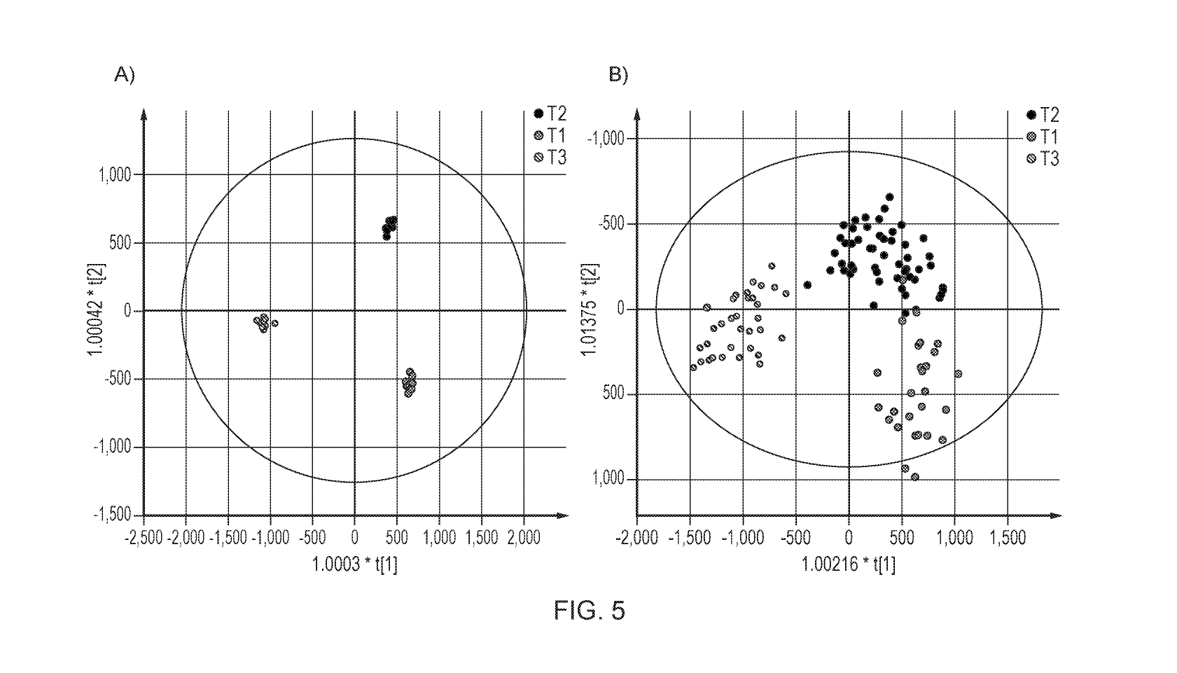

Results from the OPLS-DA model are shown in FIG. 5A (for the standard taste blend samples) and FIG. 5B (for the internally graded samples). As shown in FIG. 5A, there is a clear separation of the standard taste blends, with all samples having the T3 taste (shown in red) located in the lower left quadrant, all samples having the T2 taste (shown in green) located in the upper right quadrant, and all samples having the T1 taste (shown in blue) located in the lower right quadrant. The internally graded samples are shown in FIG. 5B with the same coloring scheme to denote taste (with each grade also being marked with an internal classifier). The various internal grades for each taste are clustered about approximately the same central positions as shown for the blends of FIG. 5A, although with more scatter (as would be expected, since they are individual, different grades of tobacco), and with some slight overlap between the tastes at the edges of the clusters. In effect, FIG. 5B shows a continuum starting in the lower left quadrant, rising into the upper central portion of the diagram, and then dropping back down into the lower right quadrant.

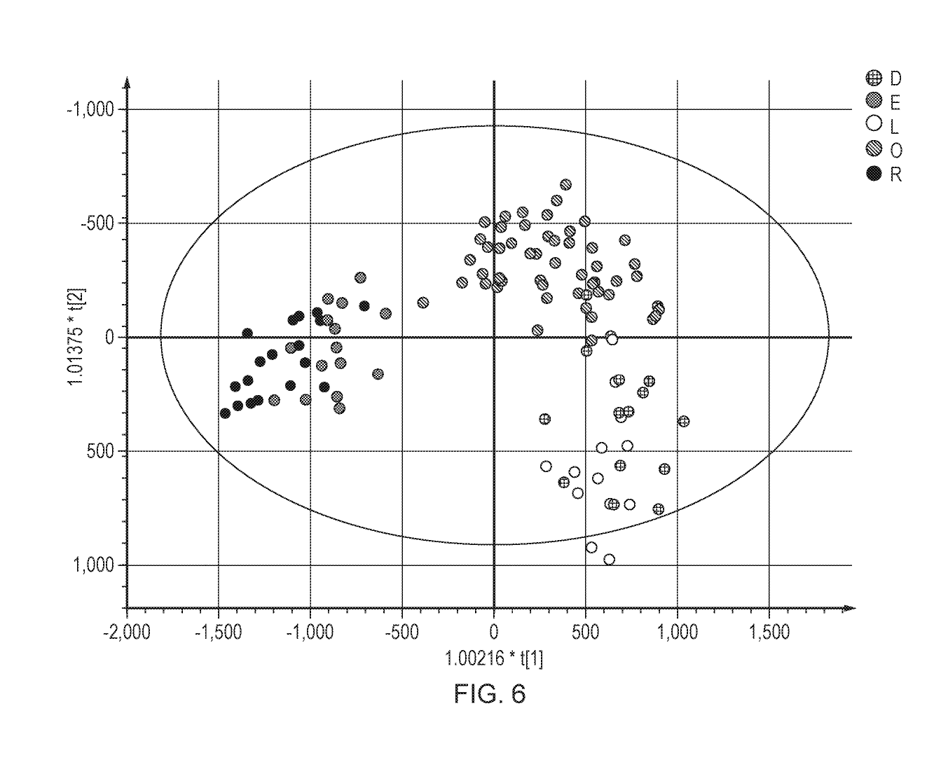

It has been found that the differentiation based on chemical constitution is strongly correlated with the blend color, which is one of the most important organoleptic characteristics employed in internal grading of Virginia tobacco. Thus each grade is assigned a colour from a spectrum comprising: Lemon (L)→Lemon-orange (D)→Orange (O)→Orange-mahogany (E)→Mahogany (R). FIG. 6 is the same plot as FIG. 5B, but with the circle for each grade colour-coded to indicate the assigned colour from the above spectrum (rather than the taste classification of FIG. 5B). It can be clearly seen that passing along the continuum from lower left, through upper centre, and then back down to lower right, corresponds to an increasing lightening of the leaf colour, from dark (mahogany) in the lower left, through medium (orange) in the upper centre, and then light (lemon) in the lower right.

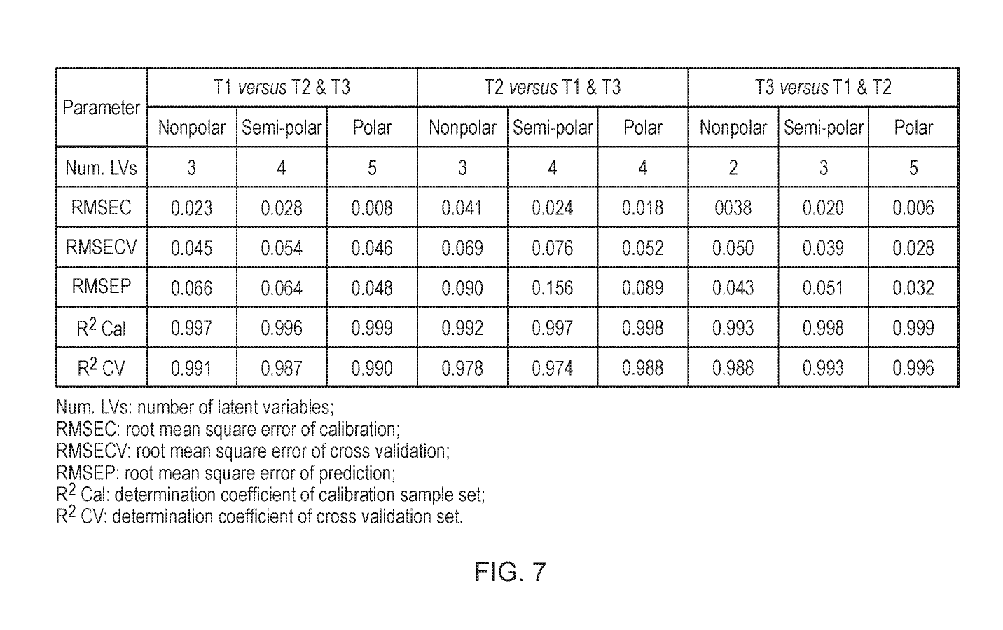

In order to obtain the chemical markers responsible for blend differentiation for the three taste blends, T1, T2 and T3, nine OPLS-DA models were generated. The relatively low root mean errors 0.16) resulting from the cross validation (RMSECV) and prediction (RMSEP), combined with a high coefficient of cross validation (R² CV≥0.97), indicate that the experimental data are properly fitted to the proposed OPLS-DA models without evidence of data overfitting, as shown in the table of FIG. 7. The models were also verified by performing suitable permutation tests.

From the blend analyses, 96 chemical markers were putatively identified. In summary, the T1 taste showed higher contents of polyphenols, carbohydrates, and lipids, whereas the T3 taste showed higher contents of nitrogen compounds (amines, amides, aminoacids, and nucleosides) and aldehydes, esters, ketones, and alcohols, in general. The T2 taste showed intermediate chemical characteristics when compared to the T1 and T3 tastes (which corresponds, for example, to the intermediate position of the T2 taste grades in the plot of FIG. 5B).

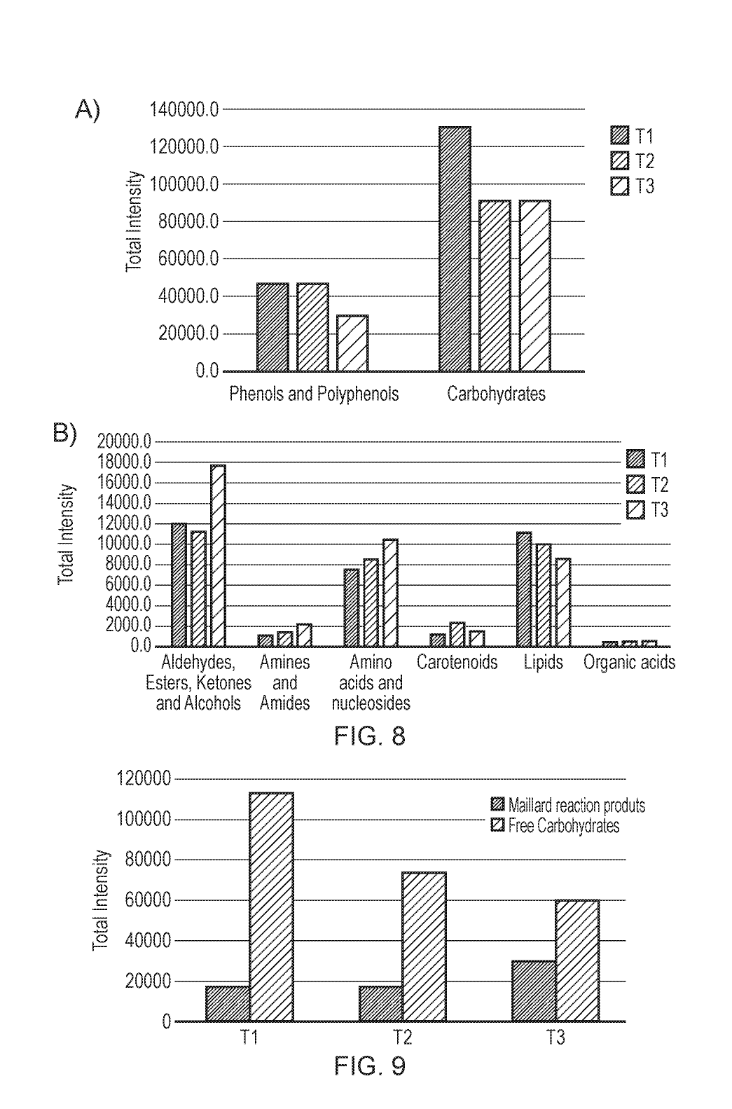

FIG. 8 illustrates these results, showing the content level of various chemical families for each of the three tastes. For each chemical family, the T1 taste is shown to the left (in blue), the T2 taste is shown in the centre (in green), and the T3 taste is shown to the right (in red). FIG. 8 is split into two plots, the left-hand plot (denoted A) representing the major chemical components, and the right-hand plot (denoted B) representing the more minor chemical components (note the reduced intensity scale of plot B compared with plot A).

Considering the results in more detail, the T1 taste showed the highest content of free carbohydrates, such as hexose (fructose, glucose, and galactose), disaccharides (lactose and sucrose) and trisaccharides like raffinose. In addition, the T1 taste showed the highest content of lipids, such as fatty acids (arachidonic acid, 5,8,11-eicosatrienoic acid, and olean-12-en-29-oic acid, 3-hydroxy-11-oxo-, (3,20)), and tri- and diglycerides. This behaviour seems to be related with blend ripeness at harvest, since the T1 taste is obtained from the flue-cure of unripe Virginia tobacco. On the other hand, the T3 taste showed the highest content of deoxyfructosazines (2,5 and 2,6), products of a Maillard reaction between free carbohydrates and ammonia. The increase of Maillard products corresponded with a decrease in the free carbohydrates (i.e. an inverse correlation. This is illustrated by the plot of FIG. 9, which shows the content level of Maillard reaction products and free carbohydrates for each of the 3 tastes T1, T2 and T3.

In contrast, higher contents of Amadori compounds (N-(1-Deoxy-1-fructosyl)proline, N-(1-Deoxy-1-fructosyl)histidine, and N-(1-Deoxy-1-fructosyl)alanine) were found in the T1 taste. Amadori compounds are products of a Maillard reaction between free carbohydrates and amino acids (Davis & Nielsen 1999; Shigematsu et al. 1977; Rodgman et al. 2013). Recognizing that the Amadori compounds are subject to low temperature degradation, this is likely to have contributed to their degradation from tobacco curing and ripeness (Davis & Nielsen 1999; Shigematsu et al. 1977). Probably, the higher maturation time and curing time for T3 taste (compared with T1 taste) increase the deoxyfructosazine contents while decreasing the content of Amadori compounds.

Significant decreases in caffeoylquinic acid derivatives (chlorogenic acid, neochlorogenic acid, glucocaffeic acid, chlorogenoquinone, and trans-p-Coumaric acid 4-glucoside) and flavonoids (rutin and kaempferol 7-galactoside 3-rutinoside) were found in the T3 taste, which seem to be related to the farming and curing of Virginia tobacco. Similarly, the higher content of nitrogen compounds found in the T3 taste compared to the T1 and T2 tastes could also be related to the different ways of farming and curing Virginia tobacco.

### c) Smoke Analysis

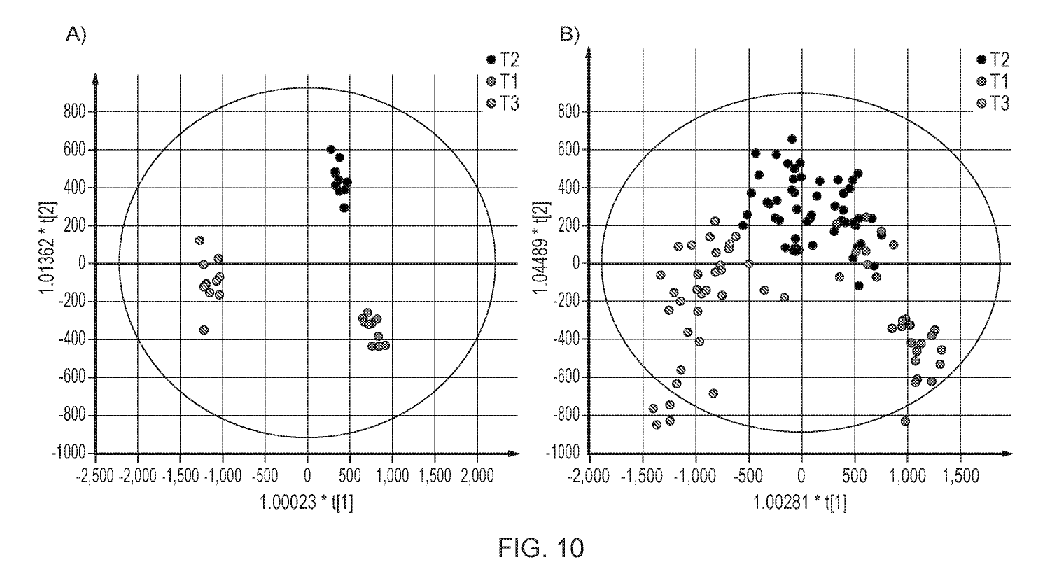

Just as for blend analysis, smoke analysis provided a clear differentiation between the three taste blends (T1, T2 and T3), as well as between the different internal grades of Virginia tobacco. Results from the OPLS-DA model for the smoke analysis are shown in FIG. 10A (for the taste-classified samples) and FIG. 10B (for the internally graded samples). These Figures are analogous to FIGS. 5A and 5B for the leaf extracts. Again, as shown in FIG. 10A, there is a clear separation of the taste blends, with all samples having the T3 taste (shown in red) located on the left-hand side, all samples having the T2 taste (shown in green) located in the upper right quadrant, and all samples having the T1 taste (shown in blue) located in the lower right quadrant. Similarly, the internally graded samples are shown in FIG. 10B with the same coloring scheme to denote taste (with each grade also being marked with an internal classifier). The various internal grades for each taste are clustered about approximately the same central positions as shown for the blends of FIG. 10A, although with more scatter, and with some slight overlap between the taste at the edges of the clusters. In effect, FIG. 10B (like FIG. 5B) shows a continuum starting in the lower left quadrant, rising into the upper central portion of the diagram, and then dropping back down into the lower right quadrant.

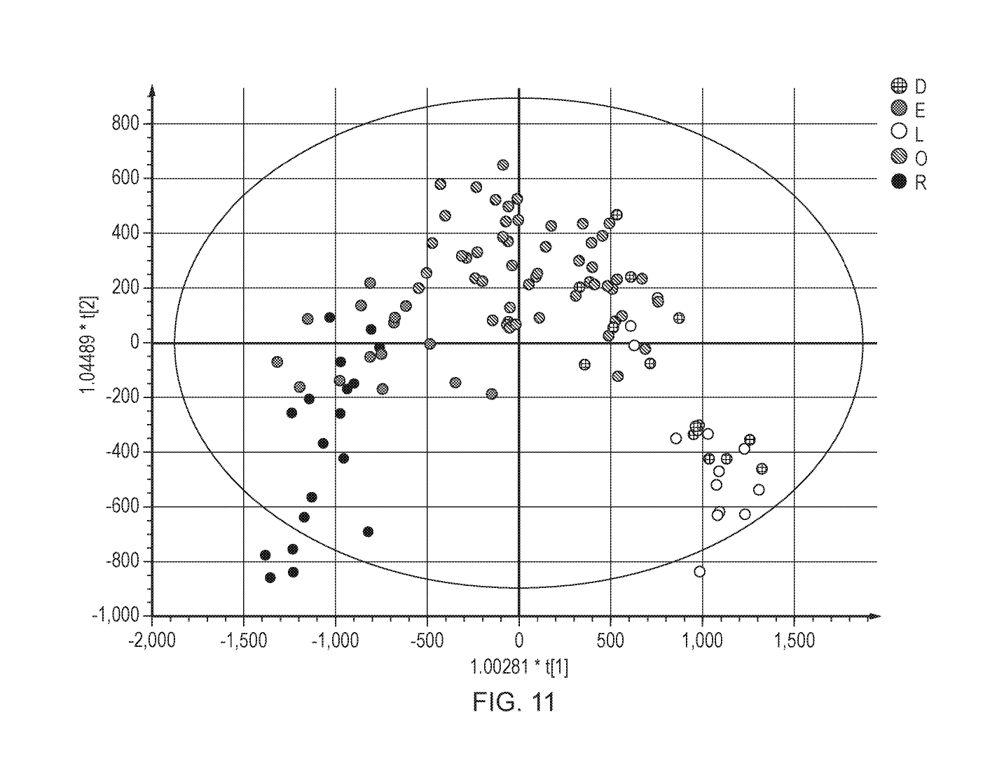

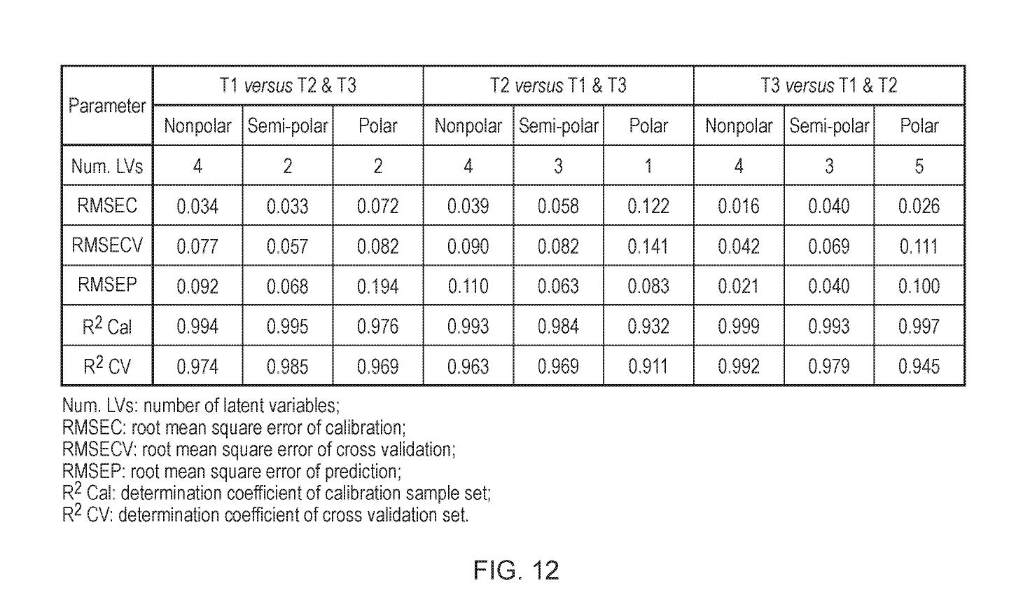

Again it has been found that the differentiation based on chemical constitution is strongly correlated with the blend color. Thus each grade is assigned a colour from the spectrum comprising: Lemon (L)→Lemon-orange (D)→Orange (O)→Orange-mahogany (E)→Mahogany (R). FIG. 11 is the same plot as FIG. 10B, but with the circle for each grade colour-coded to indicate the assigned colour from the above spectrum (rather than the taste classification of FIG. 10B). It can be clearly seen that passing along the continuum from lower left, through upper centre, and then back down to lower right, corresponds to an increasing lightening of the leaf colour, from dark (mahogany) in the lower left, through medium (orange) in the upper centre, and then light (lemon) in the lower right.

As with the blend analysis, in order to identify the chemical markers responsible for smoke differentiation into the 3 taste categories T1, T2 and T3, nine OPLS-DA models were generated. The relatively low root mean errors 0.20) resulting from the cross validation (RMSECV) and prediction (RMSEP), combined with a high coefficient of cross validation (R² CV≥0.97) indicate that the experimental data are properly fitted to the proposed OPLS-DA models—see the table of FIG. 12. The models were also verified by performing suitable permutation tests. The higher RMSECV and RMSEP values obtained from smoke analysis (as per FIG. 12) compared with from the blend analysis (as shown in the table of FIG. 7), is believed to arise from greater variability in the smoke sample preparation (the use of a smoking machine step followed by particulate matter extraction with a Cambridge filter pad).

From the smoke analyses, 96 chemical markers were putatively identified, many which are known to have important flavour and taste characteristics. In summary, the T1 smoke showed the highest contents of lipids, organic acids, and sugar, whereas the T3 smoke showed the highest contents of amines and amides and aldehydes, esters, ketones, and alcohols. The T2 smoke showed intermediate levels of compounds when compared to the T1 and T3 tastes (which corresponds, again to the intermediate position of the T2 taste grades in the plot of FIG. 10B).

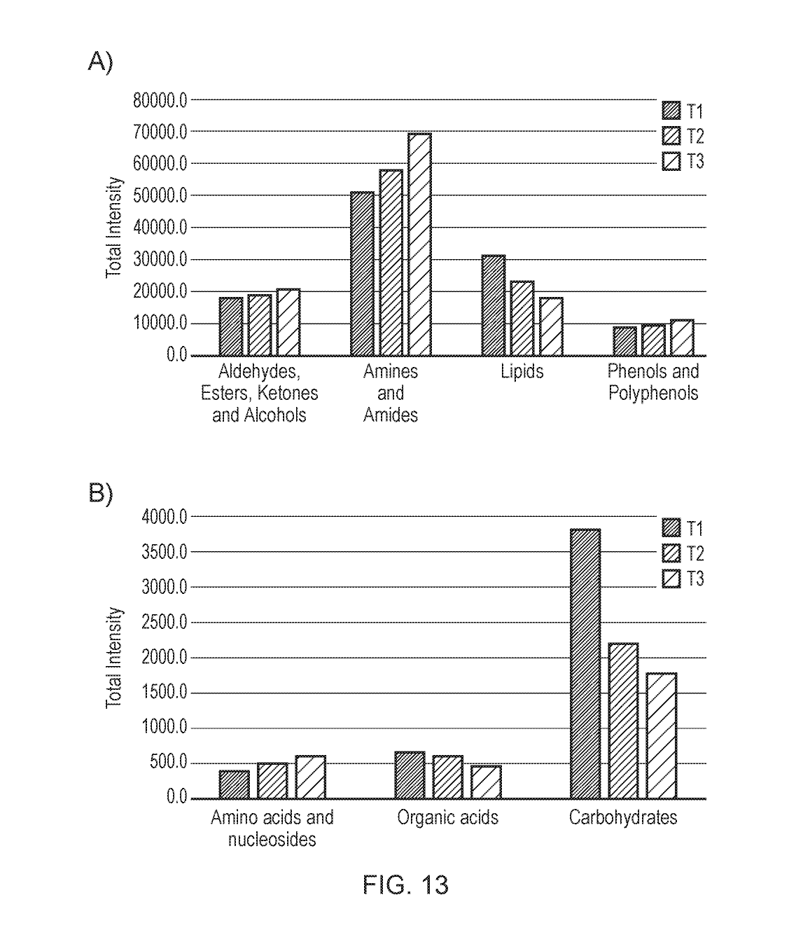

FIG. 13 illustrates these results, showing the content level of various chemical families in the smoke results for each of the three tastes. For each chemical family, the T1 taste is shown to the left (in blue), the T2 taste is shown in the centre (in green), and the T3 taste is shown to the right (in red). FIG. 13 is split into two plots, the left-hand plot (denoted A) representing the major chemical components, and the right-hand plot (denoted B) representing the more minor chemical components (note the reduced intensity scale of plot B compared with plot A).

The higher content in T1 smoke of lipids, such as fatty acids, fatty acid esters, tri- and diglycerides, seems to be derived by a direct transfer from the blend through a hydro-distillation process. Likewise, a small fraction of the free carbohydrates found in the blend also seems to be transferred into the smoke by a hydro-distillation process. However, the major fraction of free carbohydrates in the T1 blend is pyrolysed as part of the burning of the cigarette, generating 5-hydroxymethyl-furfural and other phenol compounds that are found in high concentration in T1 taste smoke (Rodgman et al. 2013).

A higher content of nitrogen compounds, mainly pyrazines, pyridines, indoles and imidazoles and pyrroles, was found in smoke from T1 tastes, and many of these compounds are known to have important flavour or taste characteristics (Rodgman et al. 2013). It seems most likely that these compounds are generated from the pyrolysis of the Maillard products, such as deoxyfructosazines, which were found in higher concentration in the FW taste blend.

### d) Blend and Smoke Correlation

In contrast to PLS and OPLS, O2PLS performs a bidirectional analysis, i.e. X H Y; therefore, X can be used to predict Y, and Y can be used to predict X. O2PLS allows the partitioning of the systematic variability in X and Y into three parts: the X/Y joint predictive variation; the variation in X which is orthogonal to Y; and the variation in Y which is unrelated to X (Trygg, 2002).

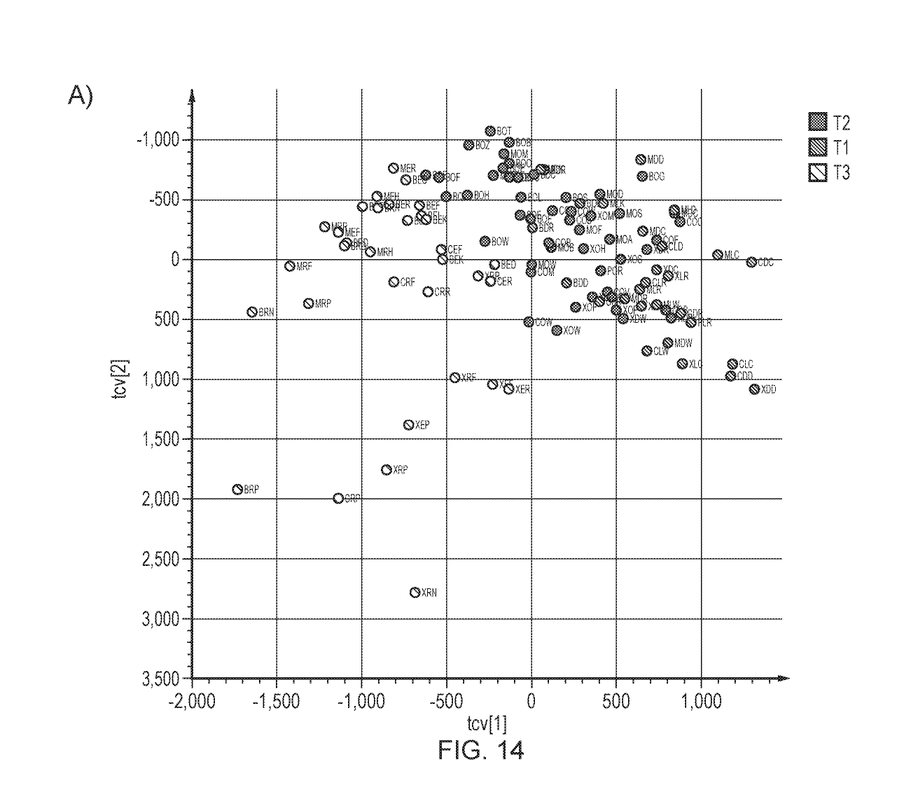

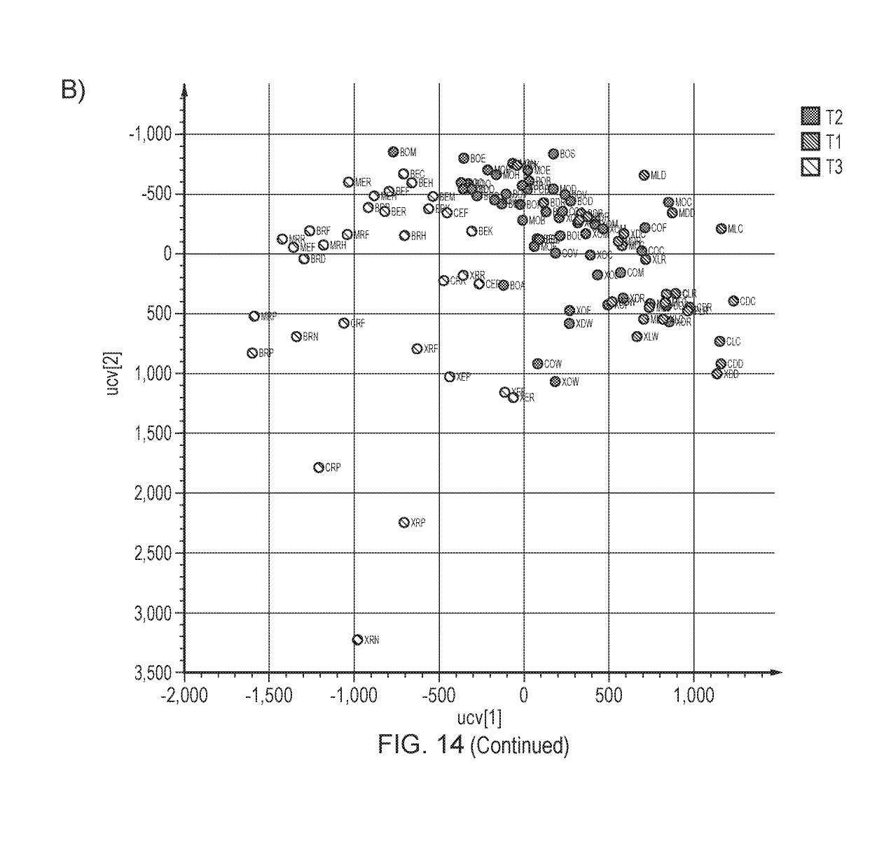

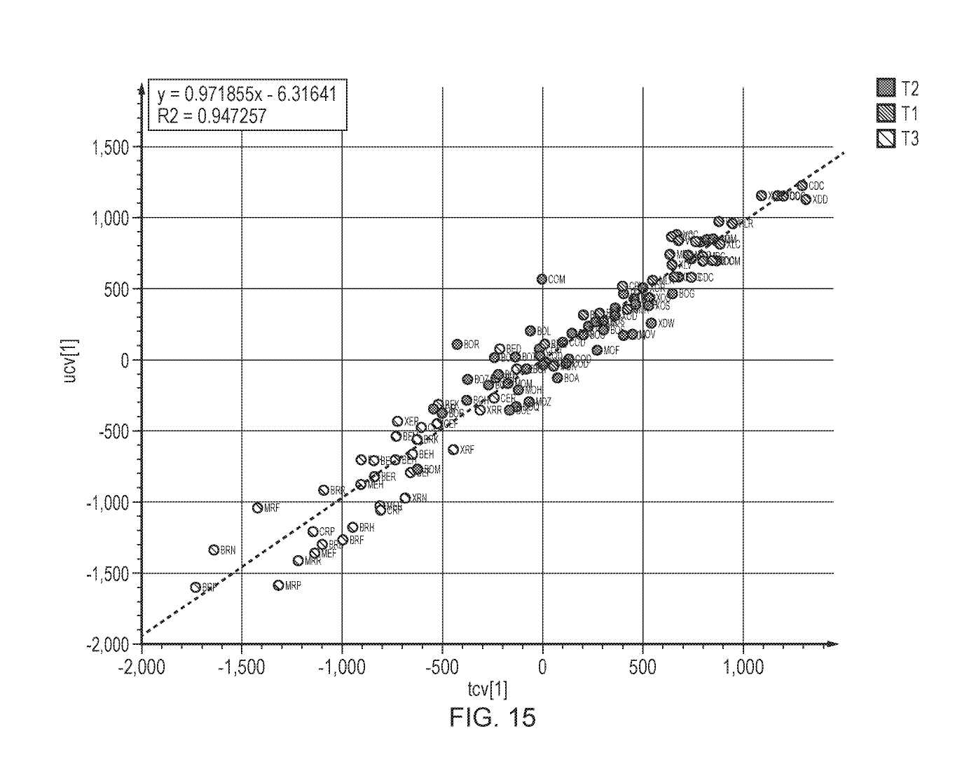

As shown in FIG. 14, the O2PLS model was applied firstly to generate separate representations of the blend samples (A—left), representing the X matrix, and smoke samples (B—right), representing the Y matrix, for the internal grades of Virginia tobacco. A correlation was then performed using the O2PLS model, based on total chemical constitution, between the X matrix from the blend results (t[1] axis) and the Y matrix from the smoke results (t[2] axis), as shown in FIG. 15.

It can be seen from the individual O2PLS plots of FIG. 14 for the blend and smoke results respectively, it is possible to see differentiation between the T1, T2 and T3 tastes of the internal grades (with T2 again lying in an intermediate position between T1 and T3). Moreover, the same sample grades (as indicated by the individual lettering/labelling for each point) show very similar scores in the blend and smoke analysis of FIGS. 14A and 14B respectively. This already indicates that there is likely to be a suitable correlation between the blend and smoke results. This expectation is confirmed by the high coefficient (R²>0.94) found in the O2PLS global correlation plot of FIG. 15. Consequently, it is found that the total blend chemical composition reflects (and determines) the total smoke composition, and so it is possible to predict the sensory characteristics of the smoke (e.g. flavour and taste) from blend analysis (and vice versa).

### e) Conclusion

The metabolomics analysis described herein provides a chemometric-based strategy that allows an untargeted chemical characterization of tobacco blend (leaf) and smoke (or vapour). Moreover, approximately two hundred chemical markers have been identified as primarily responsible for the differentiation between three different tastes of Virginia tobacco. The major chemical variations observed within the range of Virginia tobacco seems to be related to farming and curing procedures, such as reflected in the higher contents of carbohydrates and nitrogen compounds, respectively, as found in two different blends of Virginia tobacco. Accordingly, the harmonization of the farming and curing procedures seems to be highly desirable for enhancing the homogeneity of the Virginia tobacco taste.

In addition, a robust global correlation (R²>0.94) has been found between the total chemical composition of (i) blend and (ii) smoke, thereby indicating a clear relationship between these different samples. Consequently, the individual tastes and sensory properties of the smoke produced by different grades of Virginia tobacco can be predicted from a blend analysis. In particular, the sensorial characteristics of smoke (or vapour) can be predicted from a blend chemical analysis combined with a chemometric approach, thereby confirming the importance of the approach described herein to plant breeding, the consistency of the crop, taste differentiation, and tobacco grading.

### 5) Further Methodology

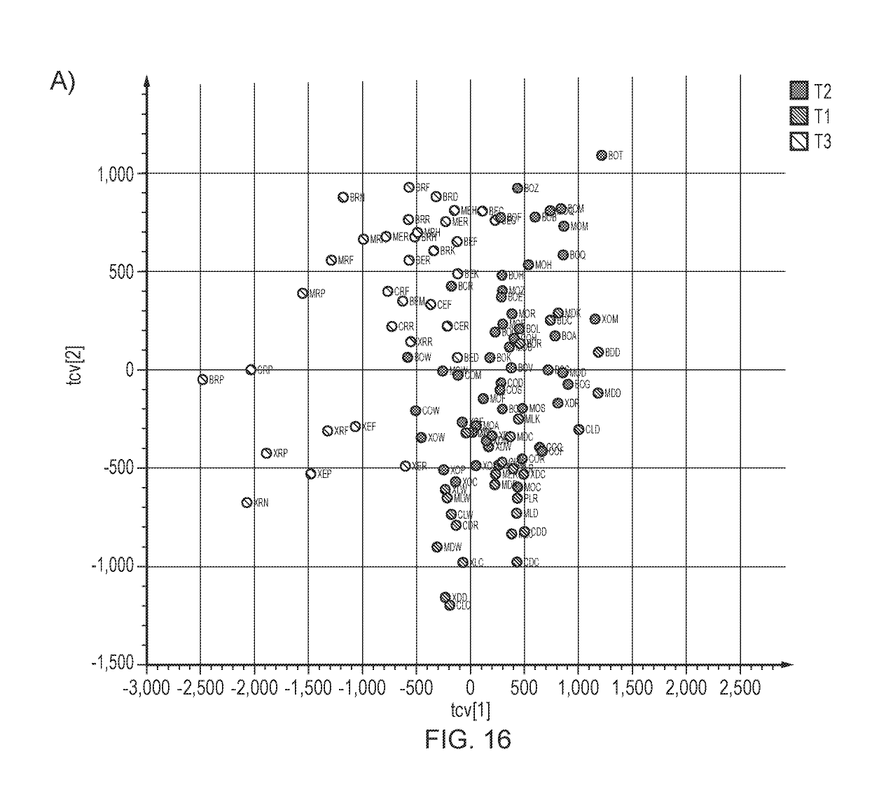

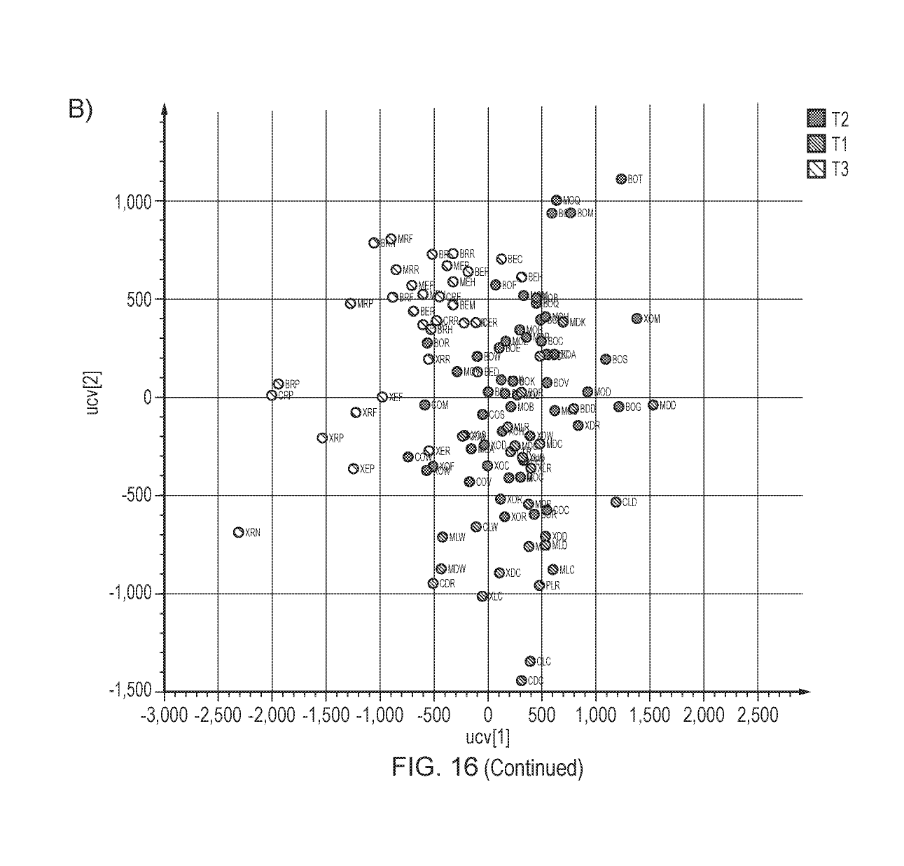

A further investigation was performed to compare the results from (i) using UPLC-HDMS^{E} as the mechanism for performing the chemical analysis, with (ii) using instead a high-throughput screening (HTS) methodology with flow injection analysis (FIA) coupled to a high-resolution mass spectrometry detection system (HTS-FIA-HRMS). As shown in FIG. 16, an O2PLS model was applied firstly to generate separate representations of the results from the UPLC-HDMS^{E} analysis (A—left), denoted as the X matrix, and from an HTS-FIA-HRMS analysis (B—right), denoted as the Y matrix, for the internal grades of Virginia tobacco. A correlation was then performed using the O2PLS model, based on total chemical constitution, between the X matrix from UPLC-HDMS^{E} analysis results (tcv[1] axis) and the Y matrix from the HTS-FIA-HRMS analysis (ucv[1] axis).

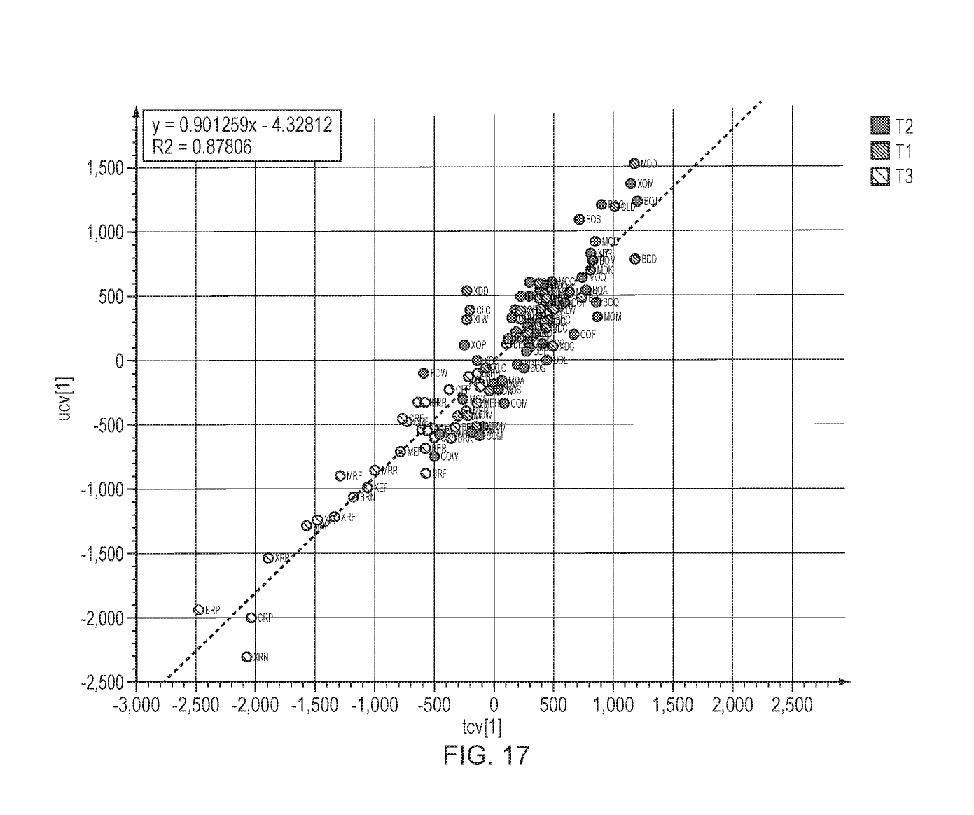

Closely matching sets of results were obtained by using the two different methodologies, UPLC-HDMS^{E} and HTS-FIA-HRMS, as can be seen from the similar distributions of samples in the plots of FIG. 16A and FIG. 16B, respectively. This indicates that there is likely to be a suitable correlation between the UPLC-HDMS^{E} and HTS-FIA-HRMS methodologies, as confirmed by the high coefficient (R²=0.88) found in the O2PLS global correlation plot of FIG. 17.

Consequently, the HTS-FIA-HRMS methodology can be seen from FIGS. 16 and 17 to provide a chemical profile which is analogous to that obtained from UPLC-HDMS^{E} methodology. Moreover, the use of HTS-FIA-HRMS provides a significant increase in the analytical capability in terms of throughput (typically by a factor of about 25). Therefore, the increase in the analytical throughput obtained from HTS-FIA-HRMS methodology supports the use of this technology across a wider range of applications.

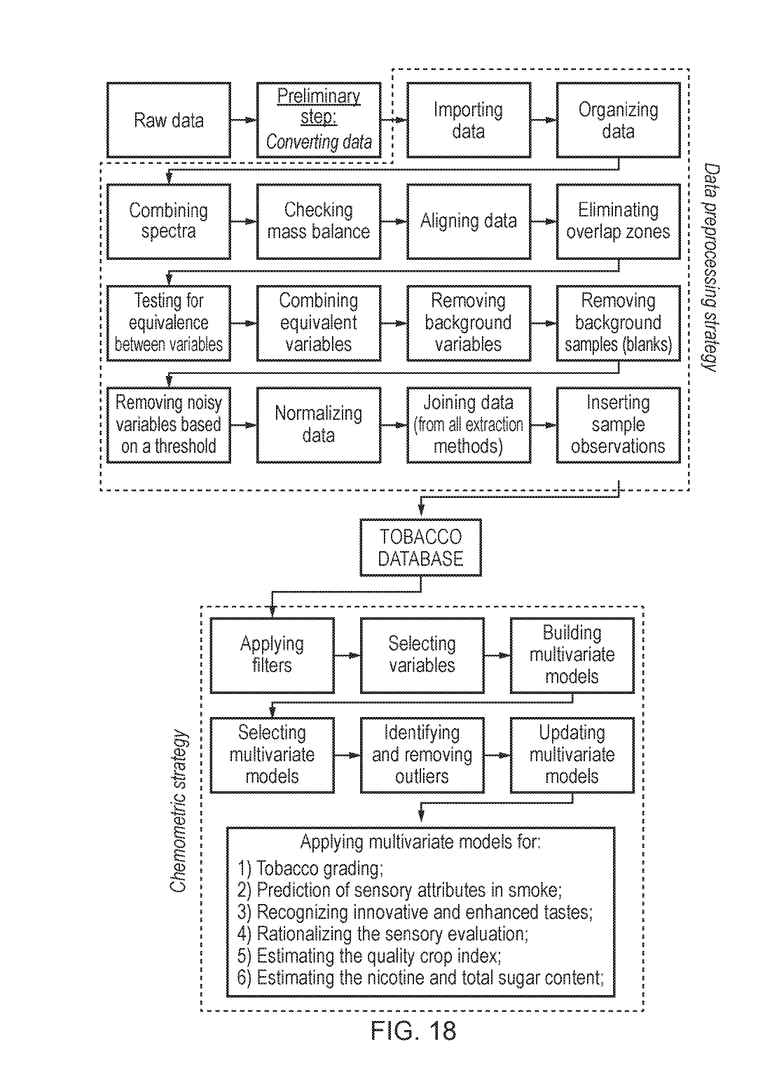

FIG. 18 is a detailed flow-chart illustrating a step-by-step approach for another implementation of the chemometric and metabolomics approach described herein. This approach is particularly suited for processing the results from thousands of samples (in a single batch), where the results have been generated, as a large and complex data set, by a detection system that uses the HTS-FIA-HRMS methodology described above. For each step shown in FIG. 18, a procedure (or procedures) have been implemented using the MATLAB programming language. This strategy has proven to be fast and effective for combining high-resolution mass spectra, aligning the data and building resulting databases for use in all further applications, such as, tobacco grading, prediction of sensory attributes, recognizing innovative and enhanced taste, rationalization of sensorial evaluation, among others.

As described above, a bi-layer procedure was used for tobacco extraction allowing the extraction of both aqueous and organic phases from the source material (leaf blend and/or smoke). This source material was taken from a range of tobacco types (not just Virginia). A high throughput screening (HTS) methodology was employed based on using an ultra-performance liquid chromatograph (UPLC) as a flow injection analysis (FIA) system, coupled to a high resolution mass spectrometer (HRMS). Two independent methods based on HTS-FIA-HRMS were applied to both extracts (aqueous and organic) in either negative or positive polarities (ESI− and ESI+), thereby resulting in four fingerprint spectra per sample in two minutes of analysis.

As shown in FIG. 18, the raw data—e.g., extension RAW for datasets acquired, without preprocessing from an ACQUITY UPLC module coupled with an SYNAPT G2-Si HDMS—both Waters, USA, are then subjected to a data conversion step, which may be performed using the MassLynx Databridge software (as described in the MassLynx 4.1 Interfacing Guide, Waters Corporation see http:/www.waters.com/webassets/cms/support/docs/71500123505ra.pdf). This conversion is performed to obtain the data in a format (NetCDF) (Network Common Data Format—a machine-independent, self-describing data format) that is compatible with the MATLAB platform (including the Bioinformatics toolbox which is part of the MATLAB platform). The data can then be imported into the MATLAB platform, where it is first organized and preprocessed according to a list (in TXT format) containing the names of the samples.

Next, the high resolution (HR) mass spectra—which contain, for example, a hundred different spectra obtained by centroid mass during a short run per sample—are combined based on the highest peak present according to a predefined delta m/z in order to obtain a single HR spectrum per sample that contains 100% of the ions combined. A check on the mass balance is then performed—in essence, it is verified that the sum of intensities of all ions present in the original spectra must be equal to the sum of intensities of all combined ions in the final spectrum per sample.

The data from the HRMS are then aligned between samples—in particular, an m/z reference vector is generated and all samples are grouped according to it. Then overlap zones are eliminated. This reflects the fact that a particular ion might be combined with either one specific reference ion or its neighbor, if the difference between them is close to the delta m/z threshold. The particular ion must be considered only once, when the difference between this particular ion and each one of the reference ions has the smallest value. The processing of FIG. 18 then proceeds to testing for equivalence between variable by comparison with its nearest neighbour to be sure that it is a unique variable, and any equivalent variables can are combined (or the redundancies are removed). Note that this elimination of data overlap and equivalence is important, otherwise there may be interference with the results of the multivariate models used in the chemometric strategy.

Next, background variables are removed, based on the contribution of the variables present in the background samples (blanks). Initially, a vector is generated containing the mean values for each variable considering all samples. Then another vector is generated containing the difference between the data from the blank and the first vector calculated, whereby the variables with positive results represent the background. This step is performed for each blank sequentially. The background samples (blanks) themselves can now be removed.

Noisy variables are now removed based on a threshold intensity, such that all variables present with intensities below this threshold are eliminated (as being too close to the noise level). However, this removal is performed only it is true for all samples per variable (i.e. all samples have the variable below the threshold)—otherwise, the full information is preserved. Next the data are normalized by using a predefined factor where each row of the matrix is divided by the quotient between the sum of intensities of all variables (per row) and this factor. This normalization is performed to improve the reproducibility of the spectra.

The processing of FIG. 18 now proceeds to join data from all extraction methods. For example, if two extracts (aqueous and organic) obtained (as illustrated in the FIG. 2), and each one is analysed in two modes of ionization (negative and positive) to detect the maximum number of compounds in the tobacco, the above global data preprocessing strategy is applied independently for all four datasets. At this step, these four data sets are then placed side-by-side in a single matrix.

Various sample observations are now inserted, involving various information for each sample, such as name, precedence, features, crop year, sensory attributes, etc.). This then results in the final tobacco database containing thousands of samples ready to be used in the multivariate models. These samples may represent a very wide range of tobacco types, including flue-cured (Virginia), air-cured (Burley and “Galpão Comum”) and sun-cured (Oriental), from several crops.

Once the tobacco database of FIG. 18 has been generated, a chemometric analysis can now be performed. Firstly, the data may be segregated by using one or more filters, such as crop year, tobacco grade, tobacco type, etc. Variables are now chosen based on the selectivity ratio (parameters available on the PLS toolbox) for each variable—this represents the power of prediction (discrimination) of each variable in a regression or classification model, according to Rajalahti et al. (2009).

The selected variables are now used to build several multivariate models, based on each set of selected variables. The objective here is to find an optimal model which is achieved according to the misclassification rates found from discriminant analysis and according to the mean squared prediction errors for regression models. This optimal model is then selected and evaluated in order to identify outliers (based on their residuals); these are then removed from the datasets. This allows the multi multivariate models to be updated by using the new datasets (without the outliers) to build the regression or classification models which are then available to be applied to tasks such as tobacco grading, prediction of sensory attributes in smoke, etc.

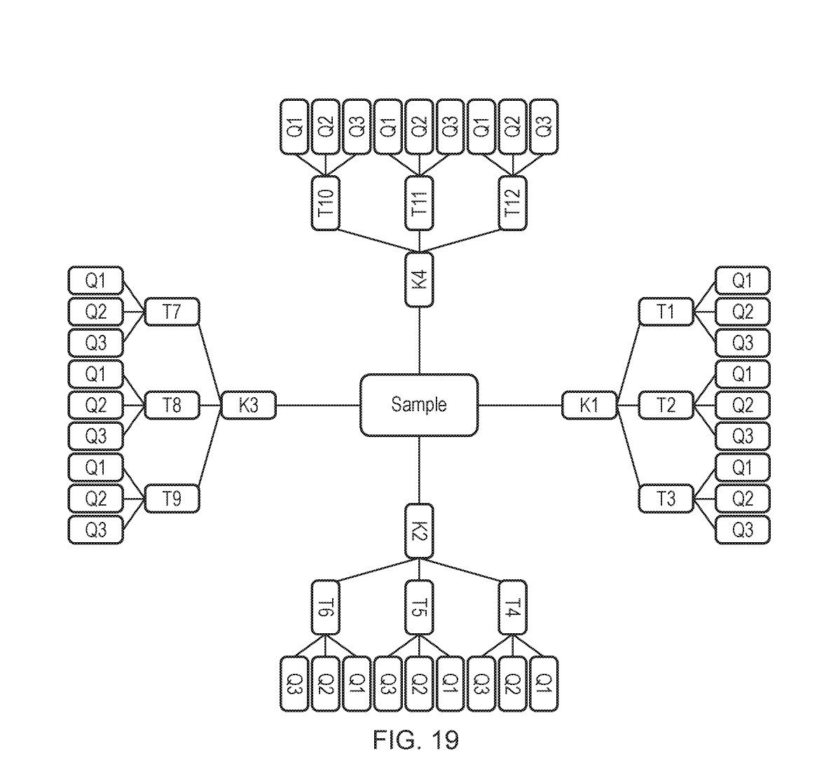

Tobacco grading represents one such example of the application of the multivariate models. In one particular implementation, the tobacco is graded with respect to tobacco type (four kinds: K1, K2, K3, K4), tobacco taste (twelve tastes: T1 to T3 for K1, T4 to T6 for K2, T7 to T9 for K3, T10 to T12 for K4) and quality (Q1: high, Q2: medium, Q3: low) based on the chemical composition of the tobacco. The association between the sensory characteristics (such as taste) and the chemical composition of tobacco samples present in the database allows multivariate models (OPLS-DA) for determination of each characteristic. These models may be built in the form of a hierarchical decision tree diagram, such as illustrated in FIG. 19. In particular, it will be observed that in FIG. 19, the model categories for taste are dependent on the tobacco type and the models for quality are similarly dependent on the tobacco taste. For example, the right-hand portion of FIG. 19 shows the hierarchical decision tree diagram for tobacco grading considering a single type of tobacco (K1) having tastes T1, T2 and T2. Q1, Q2 and Q3 then represent the different levels of quality for the tobacco for each of these three tastes. The other portions of FIG. 19 present decision trees for other types of tobacco, such as K2, which is shown as associated with tastes T4, T5 and T6, each of which again has 3 different levels of quality, and likewise for the tobacco types K3 and K4.

Another application of the tobacco database shown in FIG. 18 is for the prediction of sensory attributes in smoke. A number of sensory attributes may be selected, such as dryness, bitterness and sweetness. Such attributes are generally assessed by a panel of human experts according to a suitable scale, in which (for example), each attribute can vary from 0 (absence of sensation) up to 10 (highest intensity). Accordingly, independent calibration models (based on OPLS) can be built from the tobacco database to allow prediction of the sensory attributes in smoke based on the chemical composition of air-cured (Burley) and flue-cured (Virginia) tobaccos.

The approach described herein is not only able to process a large number of samples but is also able to characterize a tobacco based on its chemical composition. Based on this principle, it becomes possible to predict the type, taste and quality of tobacco for unknown samples, as well as various sensory attributes that are relevant to the smoke evaluation. Various other applications of this facility (now and in the future) include:

assessing a crop of tobacco prior to purchase to determine whether or not to purchase—e.g. whether it will provide the desired sensory characteristics for a particular product.
controlling the growing environment and post-harvest processing of a crop—for example, if a crop is found to have some deficiency regarding desired sensory characteristics, it may be able to rectify or compensate for this deficiency, e.g., by some post-harvest processing. Similarly, the analysis may indicate when a crop is ready for harvest (because its current chemical make-up is expected to impart the desired sensory characteristics), or likewise may indicate when curing should be terminated.
controlling blending of different tobacco to achieve a blend having the desired sensory characteristics; likewise controlling manufacturing techniques, etc to ensure that a product retains the desired sensory characteristics.
controlling plant breeding programs for the identification of innovative and/or enhanced characteristics (such as taste), given an earlier and more reliable method of determining whether a given plant will provide the desired sensory characteristics.
product quality monitoring—for example, different samples of the same cigarette brand can be compared objectively to ensure that the consumer is receiving consistent sensory characteristics.
rationalizing existing techniques for sensory evaluation (and the results obtained from such techniques).
estimating the index of crop quality.
estimating the alkaloids (e.g. nicotine) and/or total sugar content of the tobacco.

### 6) Further Applications

Various implementations and applications of the present approach will now be described in more detail by way of example. In some cases, an assessment is also provided of the validity and accuracy of using the models described above for such applications.

### 1. Tobacco Grading

A tool has been developed for use in grading tobacco according to type (four kinds: K1, K2, K3, K4), taste (twelve tastes: T1 to T3 for K1, T4 to T6 for K2, T7 to T9 for K3, T10 to T12 for K4) and quality (Q1: high, Q2: medium, Q3: low), as per the decision tree of FIG. 19, based on the chemical composition of the tobacco as determined from the analysis described above. The association between the sensory characteristics and chemical composition of tobacco samples present in the database allows building classification multivariate models for determination of each characteristic. These models were built according to the decision tree diagram of FIG. 19, i.e., the models for taste are dependent on the tobacco type and the models for quality are dependent on the tobacco taste.

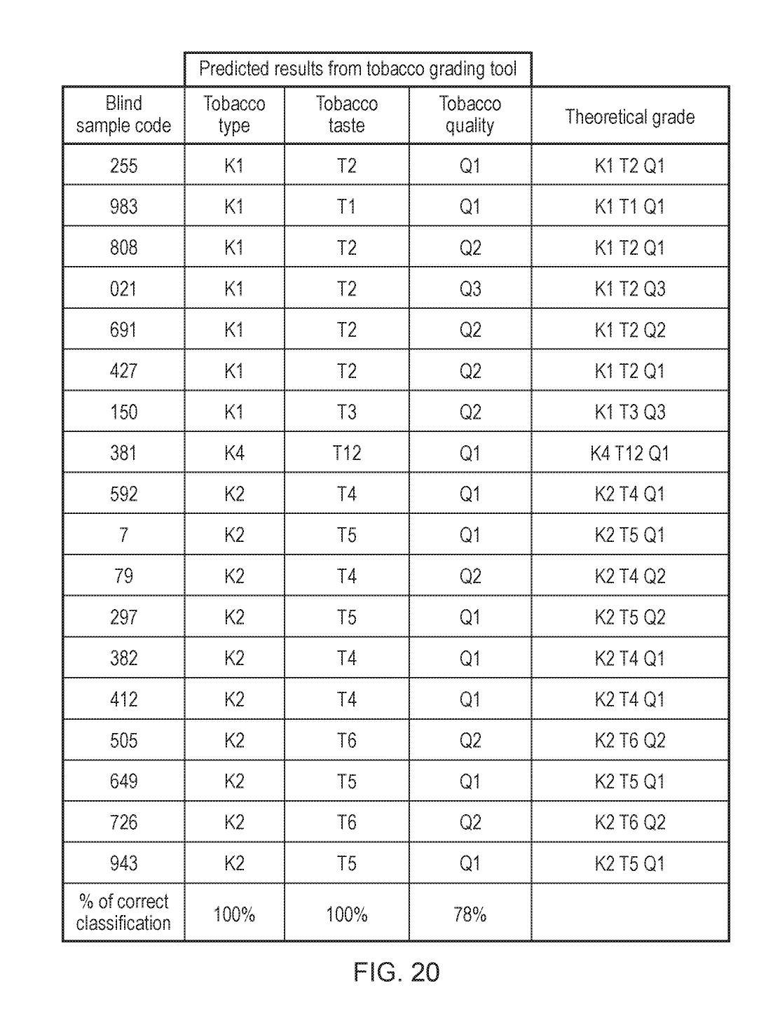

This approach has been experimentally verified, as demonstrated by the table of FIG. 20, which presents results from a blind validation of the tobacco grading tool developed from HTS-FIA-HRMS analysis. It can be seen that the grading or classification of the tobacco by type, taste and quality, as performed using the models developed from the HTS-FIA-HRMS analysis, has very good consistency with results from human experts—there is 100% match between the predicted result from the tobacco grading tool and the theoretical result (from human experts) in relation to tobacco type and taste. Furthermore, there is also a match of the predicted and theoretical tobacco quality for 14 out of 18 samples (78%), and even for the 4 samples in which there is a discrepancy in quality, this is only of one level (there are no cases in which the predicted quality and the theoretical quality differ by two levels, i.e. one is Q1 and the other Q3).

### 2. Prediction of Sensory Attributes in Smoke

The following sensory attributes of tobacco have been selected for investigation using the models described herein: impact, pitch, amplitude, irritation, balance, dryness, bitterness, sweetness, harshness. These attributes were determined for certain tobacco samples based on the sensory memory of expert panelists. Each attribute was allocated a value in the range from 0 (absence of sensation) to 10 (highest intensity). This then allowed independent calibration models to be built from the tobacco database for use in predicting the sensory attributes of smoke based on the chemical composition of air-cured (Burley) and flue-cured (Virginia) tobaccos.

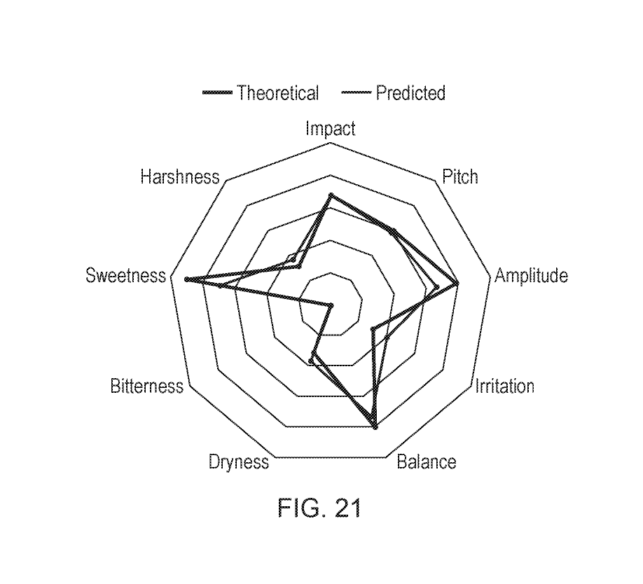

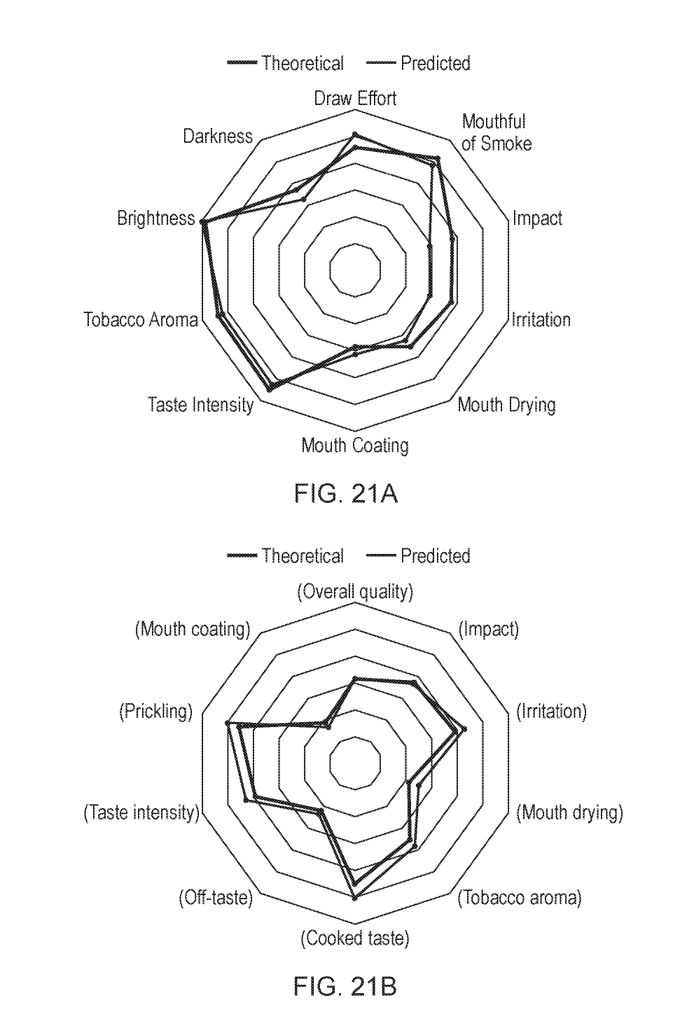

FIG. 21 illustrates a comparison between the theoretical (i.e. measured or observed) sensorial attributes of smoke from a pure tobacco sample (blue), as obtained from the human experts, and the predicted sensorial attributes (red), obtained from a tool (statistical models) developed as above from HTS-FIA-HRMS analysis. It can be seen that there is good agreement between the predicted sensorial attributes and the theoretical sensorial attributes, thereby confirming the value of this tool.

A similar approach can be extended to particular types (brands) of cigarette (combustible products), as well as to new generation products, such as tobacco heating products (heat-not-burn), electronic cigarettes (e-cigarettes) and hybrid products. For combustible products, the selected attributes were: draw effort, mouthful of smoke, impact, irritation, mouth drying, mouth coating, taste intensity, tobacco aroma, brightness and darkness. For heat-not-burn products, the selected attributes were: impact, irritation, mouth drying, tobacco aroma, cooked taste, off-taste, taste intensity, prickling, mouth coating and overall quality. In both cases, all attributes were determined by a smoking test performed by an expert panelist in a calibrated sensory panel, in which each attribute was rated on a scale ranging from 1 (lowest sensation) to 9 (highest sensation). Independent, multivariate calibration models were built by using the high-resolution mass spectrometry database to predict the sensory attributes based on smoke chemical composition for combustible products and based on vapor chemical composition for heat-not-burn products.

FIGS. 21A and 21B are analogous to FIG. 21 and illustrate the comparison between the theoretical (i.e. measured or observed) sensorial attributes of the cigarette smoke (FIG. 21A) or vapour (FIG. 21B) (blue), as obtained from the human experts, and the predicted sensorial attributes (red), obtained from a tool (statistical models) developed as above from HTS-FIA-HRMS analysis, for cigarette types or brands (FIG. 21A) and heat-not-burn products (FIG. 21B). As for FIG. 21, it can be seen from FIGS. 21A and 21B that there is good agreement between the predicted sensorial attributes and the theoretical sensorial attributes, thereby confirming the value of this tool for predicting sensory attributes.

Accordingly, the techniques described herein are useful in the context of both conventional cigarettes, which produce smoke from tobacco material, but also new generation devices, e.g. vaping devices and e-cigarettes, which produce vapour from tobacco material. For example, the approach described herein can be used to predict the sensory attributes of smoke and/or vapour produced from a given tobacco sample, thereby supporting product consistency, the development of new offerings (see below), crop management and selection decisions, and so on. It will therefore be appreciated that a tobacco sample used in the various technique described herein comprise tobacco plant material or any appropriate derivate thereof, including smoke or vapour.

### 3. Recognizing Innovative and Enhanced Tastes

A tool has been developed in order to help recognize samples with innovative and enhanced potential in new varieties of tobaccos. Firstly, a classification multivariate model has been built to predict the tobacco type (K1, K2, K3, K4) based on its chemical composition (analogous to that described above in relation to FIG. 19). Then, based on a residual analysis of predicted (new) samples, we can recognize an innovative and/or enhanced taste.

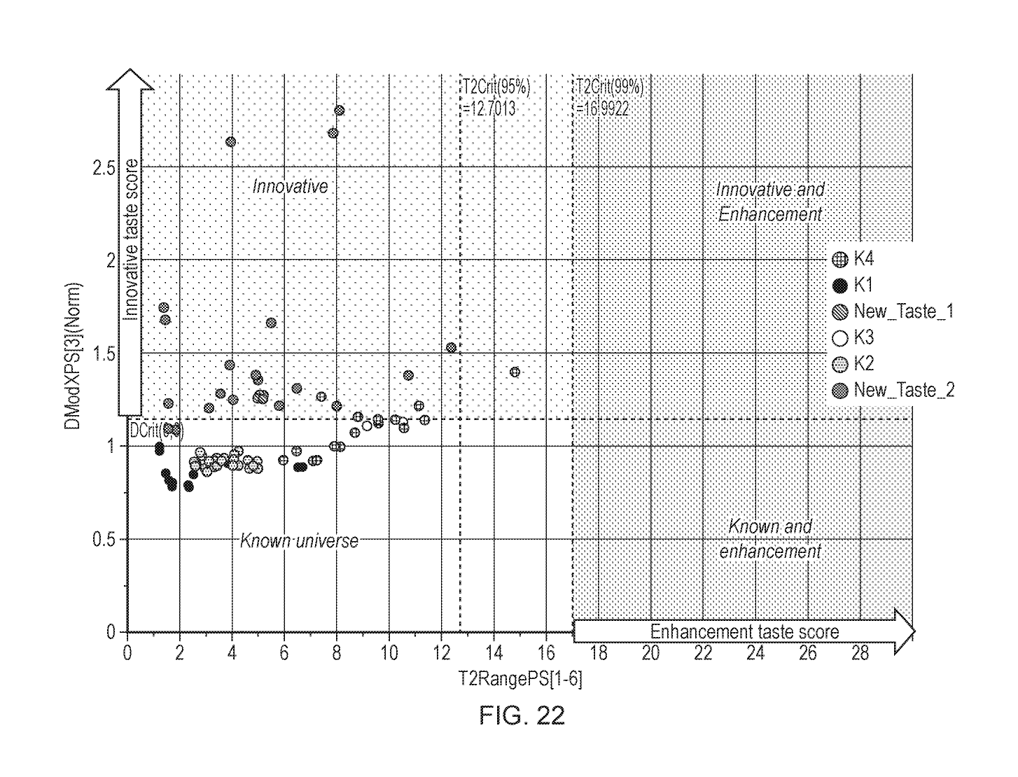

Such a residual analysis is illustrated in FIG. 22, which shows tobacco samples plotted in a two-dimensional space. The y-axis of this plot, denoted DmodX, represents the distance of an observation to the X model plane or hyperplane, being proportional to the residual standard deviation (RSD) of the X observation. Samples with a DmodX twice as large as Dcritical are regarded as moderate outliers. This indicates that these samples are different from the samples that form the known universe of tobaccos [including flue-cured (Virginia), air-cured (Burley and “Galpão Comum”) and sun-cured (Oriental), from several crops] with respect to the correlation structure of the variables (chemical composition). Samples with DmodX higher than Dcritical show differentiated chemical composition in relation to the calibration set and, consequently, higher potential as innovative taste.

On the other hand, the x-axis of this plot, corresponding to Hotelling's T² statistic, represents the distance from the origin in the model plane for each sample. Values of this statistic greater than a critical limit indicate that a sample is far from the other samples of the calibration set with respect to the selected range of components in the score space. These outlying samples represent chemical compounds having relatively higher or lower concentrations compared to their distribution in the calibration set. Therefore, samples with Hotelling's T² statistic higher than a critical limit may well show an enhanced taste in relation to the calibration set.

Moreover, with this tool we can also recognize the known basal tastes in these innovative and enhanced samples by using independent classification multivariate models to determine the 1st section (K1 to K4), tobacco type, and the 2nd section (T1 to T12), tobacco taste, in which these samples are included (analogous to the decision tree of FIG. 19).

Accordingly, FIG. 22 presents a plot of various samples that have been analysed according to the HTS-FIA-HRMS methodology. In this plot, the Y axis (vertical) represents the innovative taste score, based on the DmodX parameter, while the X axis (horizontal) represents the enhancement taste score based on Hotelling's T² statistic. Existing tobacco types (K1, K2, K3 and K4) are generally located in the “known universe” section of the plot. However, a number of samples have a DmodX value greater than Dcritical (as represented by the horizontal dashed line). Accordingly, these samples score highly for having an innovative taste. The majority of these innovative samples are allocated to New_Taste_2, although one of the innovative samples has been allocated to New_Taste_1.

### 4. Rationalizing the Sensory Evaluation

A tool has been developed to support the sensory evaluation of tobacco samples in order to differentiate tastes. The samples are clustered by the tool in accordance with their chemical similarity using hierarchical cluster analysis (HCA). The HCA is built from scores for each of multiple components obtained by principal component analysis (PCA) of the chemical composition results.

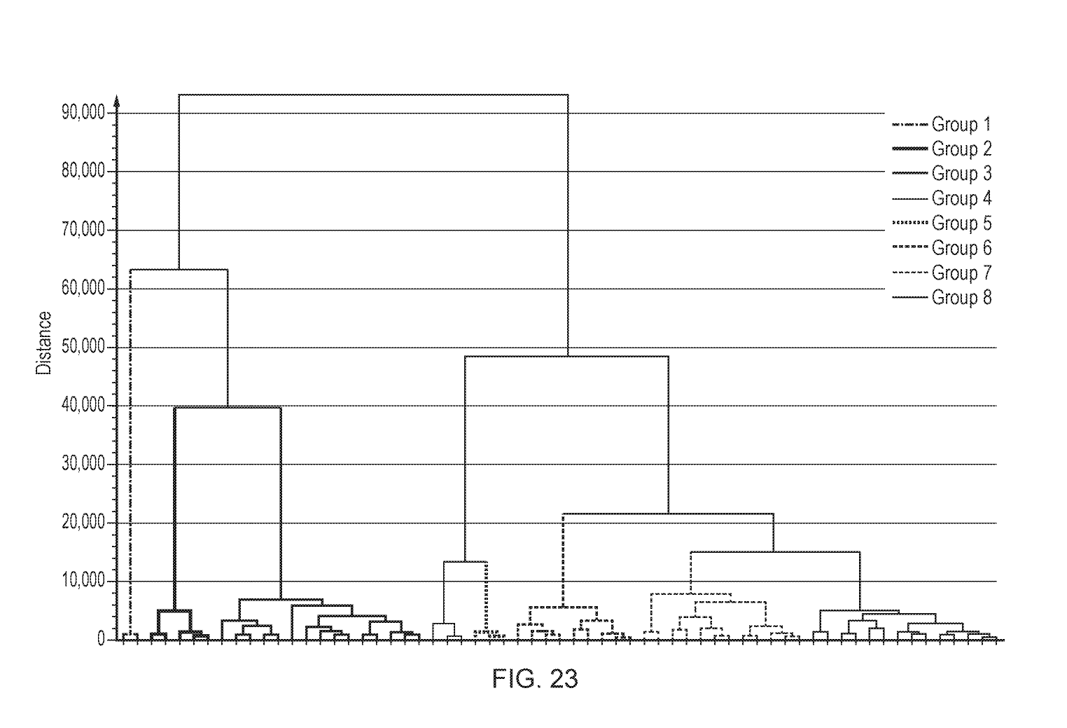

FIG. 23 shows a dendrogram formed by samples clustered in accordance with their global chemical composition determined by HTS-FIA-HRMS analysis as described above. This dendrogram provides an objective measure of whether a first tobacco is similar to, or very different from, a second tobacco. This might be useful, for example, if a given tobacco becomes unavailable or expensive (e.g. due to problems with harvest), and it is desired to identify a similar tobacco that might be used as a replacement.

### 5. Estimating the Quality Crop Index

This is a tool for use in estimating the quality crop index (QCI) based on the tobacco chemical composition. The QCI is a condensed score that represents the global sensorial quality of the smoke. The index can vary between 0 (lowest quality) and 104 (highest quality). Independent calibration models have been built using the approach described herein to predict the QCI values of air-cured (Burley) and flue-cured (Virginia) tobaccos.

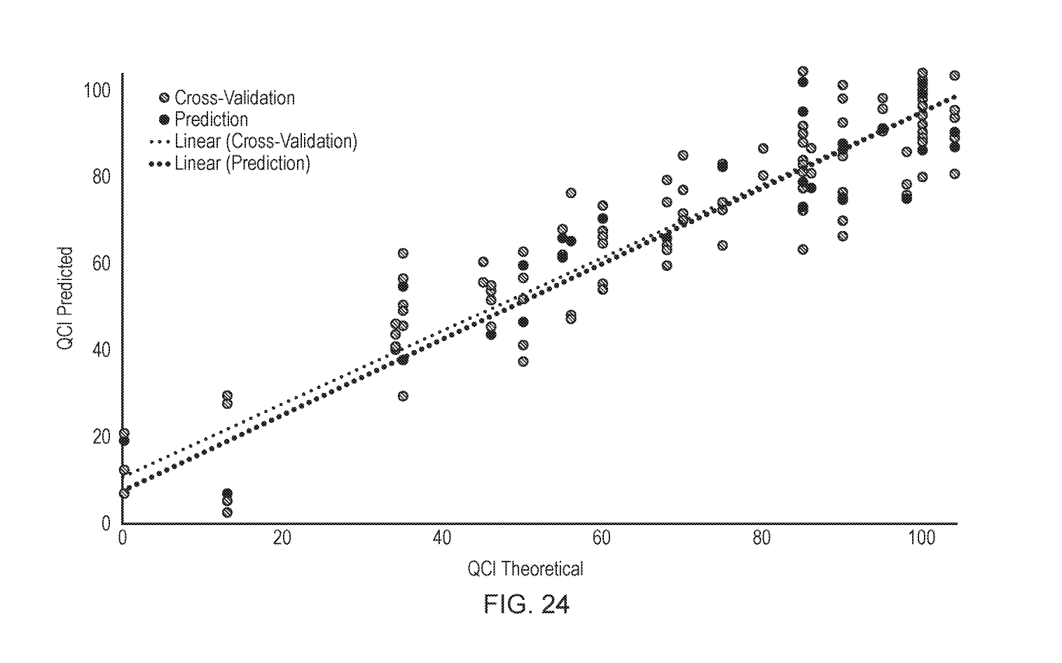

FIG. 24 illustrates a multivariate model for estimating the quality crop index (QCI) from an HTS-FIA-HRMS analysis described above. The green dots represent the cross-validation set—these represent crop samples from calibration set (used in the model building) having a known (human-rated) QCI value that were subject to the HTS-FIA-HRMS analysis described above and predicted from a multivariate model. The green dotted line then represents a linear fit between these predicted QCI values and the known, theoretical QCI values for this cross-validation set of data.

The model was then tested using a second external set of data for prediction (shown as blue dots). Again for these represent crop samples having a known (human-rated) QCI value that were subject to the HTS-FIA-HRMS analysis described above. However, the blue dots samples were not used for forming the model itself. The blue dots again illustrate QCI values predicted from the model (based on the chemical composition data from the HTS-FIA-HRMS analysis) compared with the theoretical (human-rated) QCI values. The blue dotted line then represents a linear fit between these predicted QCI values and the known, theoretical QCI values for the second set of data.

The close similarity between the linear fit for the cross-validation data (green line) compared with the linear fit obtained from the second external set of data for prediction (blue line) confirms that this model or tool, in conjunction with HTS-FIA-HRMS analysis, provides a useful mechanism for estimating the QCI for a given tobacco sample.

### 6. Estimating the Alkaloids and Total Sugar Content

This tool has been developed to estimate the alkaloids (e.g. nicotine) and total sugar content based on the tobacco chemical composition. Independent calibration models are built to predict the nicotine level (from 0 to 5%) for both air-cured (Burley) and flue-cured (Virginia) tobaccos, while the total sugar level (from 0 to 30%) is estimated only for flue-cured (Virginia) tobacco.

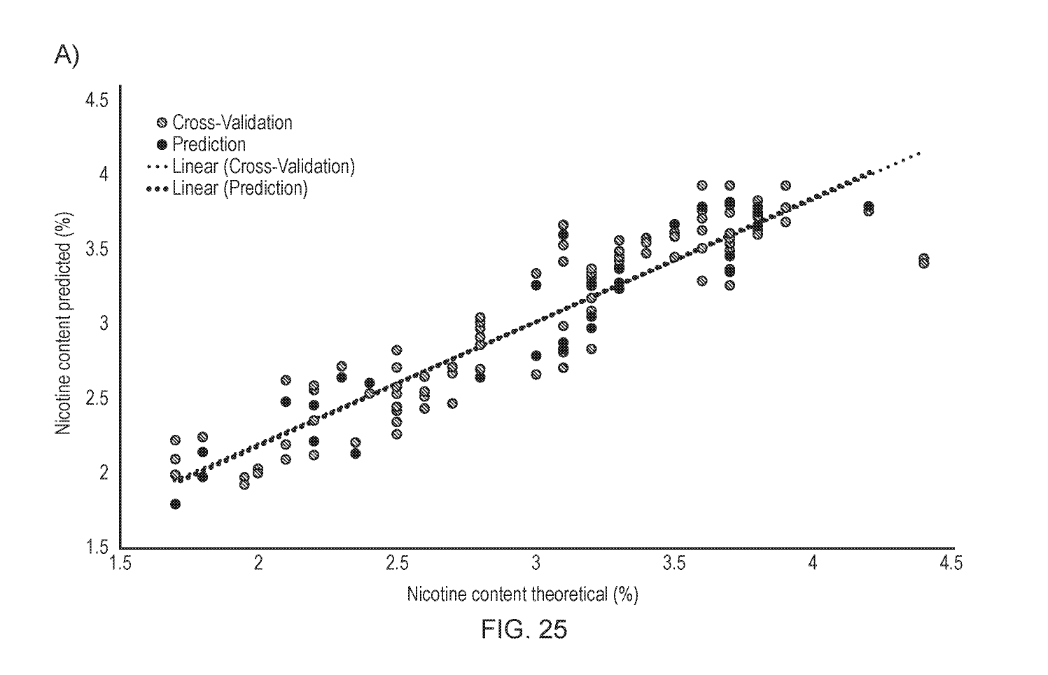

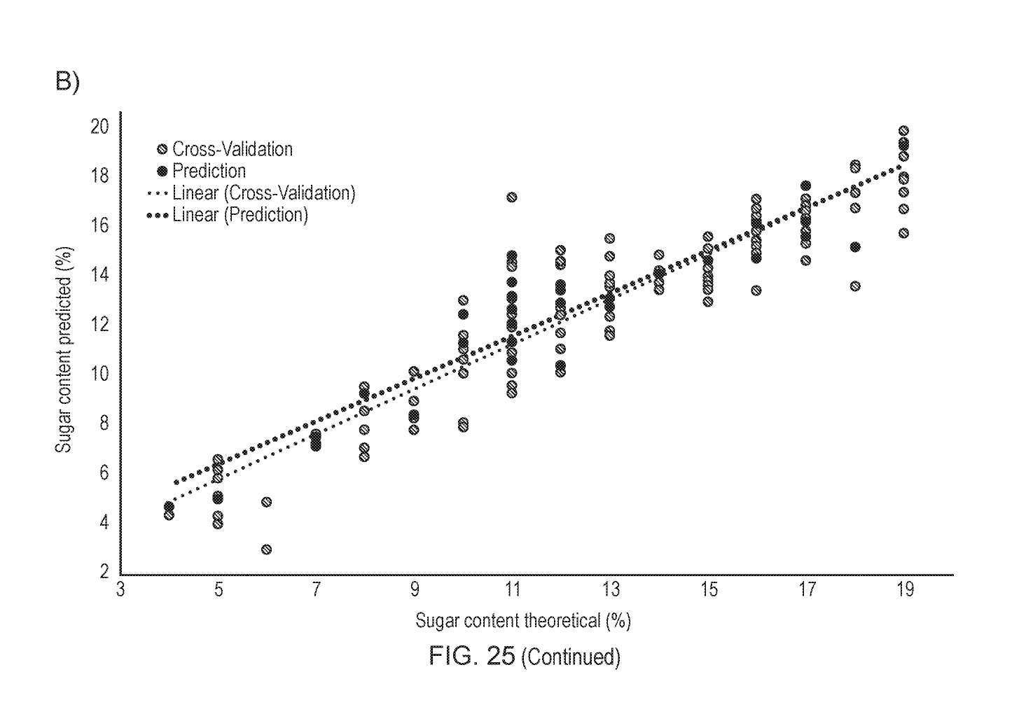

FIG. 25 illustrates the resulting fits of multivariate model for estimating the nicotine content (A—upper) and total sugar content (B—lower) from HTS-FIA-HRMS analysis. Analogous to FIG. 24, the green dots represent a cross-validation set, which represent crop samples from a calibration set used in the model building to predict nicotine/sugar content. In particular, for this set the tobacco chemical composition data is obtained as above, and this is then compared with the actual measured (theoretical) nicotine/sugar content, as per the plot of FIG. 25. The green dotted line then represents a linear fit between these predicted content values and the known, theoretical content values for this cross-validation set of data.

The model was then tested using a second external set of data for prediction (shown as blue dots). Again these represent samples having a known (measured) nicotine/sugar content that were subject to the HTS-FIA-HRMS analysis described above, but the blue dots samples were not used for forming the model itself. The blue dots again illustrate content values predicted from the model (based on the chemical composition data from the HTS-FIA-HRMS analysis) compared with the measured (theoretical) values for nicotine and sugar content. The blue dotted line then represents a linear fit between these predicted content values and the known, theoretical content values for the second external set of data.

The close similarity between the linear fit for the cross-validation data (green line) compared with the linear fit obtained from the second external set of data for prediction (blue line) confirms that this model or tool, in conjunction with the HTS-FIA-HRMS analysis, provides a useful mechanism for estimating the sugar and/or nicotine content for a given tobacco sample.

The various tools described above may be implemented using one or more computer systems provided with processors, memory, etc. In particular, the tools may be implemented using one or more computer programs executing on the computer system (s). In some cases, the one or more computer system may be general purpose machines, in other cases, they may include some special-purpose hardware—e.g. graphical processing units (GPUs) to support numerical processing. The computer programs may be provided on a non-transitory storage medium, e.g. a hard disk drive, and/or downloaded or run over a computer network, such as the Internet.

It will be appreciated that these potential uses listed above for the technology described herein are provided by way of example only, and without limitation. In conclusion, in order to address various issues and advance the art, this disclosure shows by way of illustration various embodiments in which the claimed invention(s) may be practiced. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilised and modifications may be made without departing from the scope of the claims. Various embodiments may suitably comprise, consist of, or consist essentially of, various combinations of the disclosed elements, components, features, parts, steps, means, etc other than those specifically described herein. The disclosure may include other inventions not presently claimed, but which may be claimed in future.

### 6) References

Davis D. L. & Nielsen M. T. Tobacco: Production, Chemistry and Technology (1999).
Fiehn O., Kopka J., Dormann P., Altmann T., Trethewey R. N., Willmitzer L. Metabolite profiling for plant functional genomics. Nat Biotechnol 18, 1157-1161 (2000).
Gama M. R., da Costa Silva R. G., Collins C. H., Bottoli C. B. G. Hydrophilic interaction chromatography. TrAC Trends Anal Chem 37, 48-60 (2012).
ISO 3308:2012. Routine analytical cigarette-smoking machine—Definitions and standard conditions (2012).
Kim H. K. & Verpoorte R. Sample Preparation for Plant Metabolomics. Phytochem Anal 21, 4-13 (2010).
McCalley D. V. Study of the selectivity, retention mechanisms and performance of alternative silica-based stationary phases for separation of ionised solutes in hydrophilic interaction chromatography. J Chromatogr A 1217, 3408-17 (2010).
Rajalahti T., Arneberg R., Berven F. S., Myhr K.-M., Ulvik R. J., Kvalheim O. M. Biomarker discovery in mass spectral profiles by means of selectivity ratio plot. Chemometrics and Intelligent Laboratory Systems 95, 35-48 (2009).
Rodgman A. & Perfetti T. A. The Chemical Components of Tobacco and Tobacco Smoke (2013).
Shigematsu S., Shibata S., Kurata T., Kato H., Fujimaki M. Thermal degradation products of several Amadori compounds. Agric Biol Chem 41, 2377-2385 (1977).
Shvartsburg A. A., Tang K., Smith R. D. Modeling the Resolution and Sensitivity of FAIMS Analyses. J Am Soc Mass Spectr 15, 1487-1498 (2004).
Theodoridis G., Gika H., Franceschi P., Caputi L. LC-MS based global metabolite profiling of grapes: Solvent extraction protocol optimisation. Metabolomics 8, 175-185 (2011).
Trygg J. O2-PLS for qualitative and quantitative analysis in multivariate calibration. J Chemometr 16, 283-293 (2002).
Viehland L. A., Guevremont R., Purves R. W., Barnett D. A. Comparison of high-field ion mobility obtained from drift tubes and a FAIMS apparatus. Int. J. Mass Spectrom 197, 123-130 (2000).
Villas-Boas S. G., Mas S., Akesson M., Smedsgaard J., Nielsen J. Mass spectrometry in metabolome analysis. Mass Spectrom Metabolome Anal 24, 613-646 (2005).
De Vos R. C., Moco S., Lommen A., Keurentjes J. J., Bino R. J., Hall R. D. Untargeted large-scale plant metabolomics using liquid chromatography coupled to mass spectrometry. Nat Protoc 2, 778-791 (2007).

1. A method of classifying a tobacco sample of a particular tobacco type into one of a predefined set of taste categories for that tobacco type, said method comprising:
acquiring mass spectrometry (MS) data from the tobacco sample;
identifying from the acquired MS data a plurality of chemical components and their respective content levels within the tobacco sample; and
assigning the tobacco sample to one of the predefined set of taste categories for that tobacco type based on the plurality of chemical components and their respective content levels identified within the tobacco sample, using a statistical multivariate regression model that represents a relationship between the chemical components and the taste categories.

2. The method of claim 1, wherein the tobacco sample comprises solid material derived from a tobacco leaf, and the MS data is acquired from the solid material.

3. The method of claim 2, wherein the solid material is particulate.

4. The method of claim 1, wherein the tobacco sample comprises smoke derived from pyrolysis of a tobacco leaf or vapour derived from tobacco material in a heat-not-burn device.

5. The method of any one of claims 1 to 4, further comprising performing the high definition mass spectrometry on the tobacco sample in order to acquire the MS data.

6. The method of any one of claims 1 to 5, wherein the MS data comprises high definition or high resolution mass spectrometry data (HDMS, HRMS).

7. The method of claim 6, wherein the acquired MS data comprises HDMS^{E} data using both low and high energy collision-induced dissociation for investigating precursor and product ions respectively.

8. The method of claim 6 or 7, further comprising subjecting the tobacco sample to ultra performance liquid chromatography (UPLC) as a precursor to the high definition mass spectrometry.

9. The method of claim 6, further comprising performing high-throughput screening (HTS) using a flow injection analysis (FIA) system coupled to a high-resolution mass spectrometry detection system (HTS-FIA-HRMS).

10. The method of any of claims 1 to 9, further comprising performing a multi-phase extraction on the tobacco sample using a combination of an aqueous solvent and an organic solvent.

11. The method of claim 10, wherein the multi-phase extraction includes the use of non-polar, semi-polar and polar methods.

12. The method of any of claims 1 to 11, wherein the plurality of chemical components and their respective content levels are identified within the tobacco sample using an untargeted approach.

13. The method of any of claims 1 to 12, wherein the plurality of chemical components and their respective content levels are identified by comparison with one or more libraries.

14. The method of any of claims 1 to 13, wherein statistical multivariate regression model comprises one or more statistical models based on orthogonal partial least squares (OPLS) regression and OPLS with discriminant analysis (OPLS-DA).

15. The method of any of claims 1 to 13, wherein the wherein statistical multivariate regression model statistical model utilises modelling comprising one or more multivariate supervised and/or unsupervised methods, such as Principal Component Analysis (PCA), Hierarchical Cluster Analysis (HCA), Principal Component Regression (PCR), Support Vector Machine (SVM), Artificial Neural Network (ANN), Random Forest (RF), and/or Genetic Algorithm (GA).

16. The method of claim 14 or 15, wherein the statistic model has a coefficient of cross-validation R>0.9.

17. The method of any of claims 1 to 16, wherein the statistical model differentiates between the predefined set of taste categories based on the contents of at least one of the following: polyphenols, carbohydrates, and lipids.

18. The method of any of claims 1 to 17, wherein the statistical model differentiates between the predefined set of taste categories based on the contents of at least one of the following: nitrogen compounds and aldehydes, esters, ketones and alcohols.

19. The method of any of claims 1 to 16, wherein the predefined set of taste categories can be considered as a linear sequence in which an increasing content of at least one of the following: polyphenols, carbohydrates, and lipids, corresponds to a decreasing content of at at least one of the following: nitrogen compounds and aldehydes, esters, ketones and alcohols.

20. The method of any of claims 1 to 19, wherein the predefined set of taste categories can be considered as a linear sequence related to maturation.

21. The method of any of claims 1 to 20, wherein the statistical model incorporates a correlation between the plurality of chemical components and their respective content levels within a tobacco smoke sample and the plurality of chemical components and their respective content levels within a tobacco leaf sample.

22. The method of any of claims 1 to 21, further comprising identifying the quality of the tobacco sample.

23. The method of any of claims 1 to 22, further comprising identifying the type of the tobacco sample.

24. Apparatus for classifying a tobacco sample of a particular tobacco type into one of a predefined set of taste categories for that tobacco type, said apparatus configured to:
acquire mass spectrometry (MS) data from the tobacco sample;
identify from the acquired MS data a plurality of chemical components and their respective content levels within the tobacco sample; and
assign the tobacco sample to one of the predefined set of taste categories for that tobacco type based on the plurality of chemical components and their respective content levels identified within the tobacco sample, using a statistical multivariate regression model that represents a relationship between the chemical components and the taste categories.

25. The use of the apparatus of claim 24 to predict the taste category of the tobacco sample using MS data acquired from the tobacco sample.

26. A method for generating a statistical multivariate regression model for classifying a tobacco sample of a particular tobacco type into one of a predefined set of taste categories for that tobacco type, said method comprising:
acquiring mass spectrometry (MS) data from a set of multiple tobacco samples, wherein each of said of multiple tobacco samples in said set has a known taste category;
identifying from the acquired MS data a plurality of chemical components and their respective content levels within each tobacco sample; and
generating said statistical multivariate regression model by performing a partial least squares analysis with respect to (i) the known taste category for each tobacco sample, and (ii) the plurality of chemical components and their respective content levels for each tobacco sample.

27. Apparatus for generating a statistical multivariate regression model for classifying a tobacco sample of a particular tobacco type into one of a predefined set of taste categories for that tobacco type, said apparatus being configured to:
acquire mass spectrometry (MS) data from a set of multiple tobacco samples, wherein each of said of multiple tobacco samples in said set has a known taste category;
identify from the acquired MS data a plurality of chemical components and their respective content levels within each tobacco sample; and
generate said statistical multivariate regression model by performing a partial least squares analysis with respect to (i) the known taste category for each tobacco sample, and (ii) the plurality of chemical components and their respective content levels for each tobacco sample.

28. A method of estimating at least one property of a tobacco sample comprising:
acquiring mass spectrometry (MS) data from a given tobacco sample;
identifying from the acquired MS data a plurality of chemical components and their respective content levels within the given tobacco sample; and
using a statistical multivariate regression model that represents a relationship between the chemical components and said at least one property from a population of tobacco samples to estimate said at least one property for the given tobacco sample.

29. The method of claim 28, where the at least one property comprises taste.

30. The method of claim 29, wherein the statistical multivariate regression model is further used to distinguish if the given tobacco sample comprises an innovative and/or enhanced taste.

31. The method of any of claims 28 to 30, wherein the at least one property comprises one or more of sweetness, bitterness, dryness, balance, irritation, amplitude, pitch, impact and harshness.

32. The method of any of claims 28 to 31, where the at least one property comprises a quality crop index.

33. The method of any of claims 28 to 32, where the at least one property comprises a total sugar content and/or alkaloids content (e.g. nicotine).

34. Apparatus for estimating at least one property of a tobacco sample, the apparatus being configured to:
acquire mass spectrometry (MS) data from a given tobacco sample;
identify from the acquired MS data a plurality of chemical components and their respective content levels within the given tobacco sample; and
use a statistical multivariate regression model that represents a relationship between the chemical components and said at least one property from a population of tobacco samples to estimate said at least one property for the given tobacco sample.

35. A method of classifying a tobacco sample of a particular tobacco type into one of a predefined set of taste categories substantially as defined herein with reference to the accompanying drawings.

36. Apparatus for classifying a tobacco sample of a particular tobacco type into one of a predefined set of taste categories substantially as defined herein with reference to the accompanying drawings.

37. A method for generating a statistical multivariate regression model for classifying a tobacco sample into one of a predefined set of taste categories substantially as defined herein with reference to the accompanying drawings.
